SEQUENCE LISTING (28 sequences)

SEQ ID NO: 1 - DNA, 11058 bp, synthetic construct
  source 1..11058: mol_type = other DNA; note = Sequence of the 79 exons encoding the DMD gene
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 2 - DNA, 3285 bp, synthetic construct
  source 1..3285: mol_type = other DNA; note = Microdystrophin construct 1
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 3 - DNA, 3378 bp, synthetic construct
  source 1..3378: mol_type = other DNA; note = Microdystrophin construct 2
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 4 - DNA, 3480 bp, synthetic construct
  source 1..3480: mol_type = other DNA; note = Microdystrophin construct 3
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 5 - DNA, 3639 bp, synthetic construct
  source 1..3639: mol_type = other DNA; note = Microdystrophin construct 4
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccagcctgacctagctcctggactgaccactattggagcctctcctactcagactgttactctggtgacacaacctgtggttactaaggaaactgccatctccaaactagaaatgccatcttccttgatgttggaggtacctacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtag

SEQ ID NO: 6 - DNA, 544 bp, Homo sapiens
  source 1..544: mol_type = genomic DNA; note = hTNNT2 promoter
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 7 - DNA, 756 bp, synthetic construct
  source 1..756: mol_type = other DNA; note = N-terminus-encoding sequence
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaa

SEQ ID NO: 8 - DNA, 228 bp, synthetic construct
  source 1..228: mol_type = other DNA; note = Hinge 1 encoding sequence
atgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaag

SEQ ID NO: 9 - DNA, 117 bp, synthetic construct
  source 1..117: mol_type = other DNA; note = Hinge 4 encoding sequence
gcccacagggactttggtccagcatctcagcactttctttccacgtctgtccagggtccctgggagagagccatctcgccaaacaaagtgccctactatatcaaccacgagactcaa

SEQ ID NO: 10 - DNA, 843 bp, synthetic construct
  source 1..843: mol_type = other DNA; note = CR domain-encoding sequence
acaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgc

SEQ ID NO: 11 - DNA, 978 bp, synthetic construct
  source 1..978: mol_type = other DNA; note = C-terminus-encoding sequence
actccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 12 - DNA, 273 bp, synthetic construct
  source 1..273: mol_type = other DNA; note = Partial R1-encoding sequence
tcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaa

SEQ ID NO: 13 - DNA, 357 bp, synthetic construct
  source 1..357: mol_type = other DNA; note = R1-encoding sequence
tcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacataga

SEQ ID NO: 14 - DNA, 543 bp, synthetic construct
  source 1..543: mol_type = other DNA; note = Shortened C-terminus-encoding sequence
actccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtag

SEQ ID NO: 15 - DNA, 90 bp, synthetic construct
  source 1..90: mol_type = other DNA; note = Partial R24-encoding sequence
ccgtataacctcagcactctggaagacctgaacaccagatggaagcttctgcaggtggccgtcgaggaccgagtcaggcagctgcatgaa

SEQ ID NO: 16 - DNA, 183 bp, synthetic construct
  source 1..183: mol_type = other DNA; note = Partial R24-encoding sequence
gcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaa

SEQ ID NO: 17 - DNA, 327 bp, synthetic construct
  source 1..327: mol_type = other DNA; note = R24-encoding sequence
acccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaa

SEQ ID NO: 18 - DNA, 18 bp, synthetic construct
  source 1..18: mol_type = other DNA; note = R2 connector-encoding sequence
gttttaatggatctccag

SEQ ID NO: 19 - DNA, 327 bp, synthetic construct
  source 1..327: mol_type = other DNA; note = R2-encoding sequence
gttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagac

SEQ ID NO: 20 - DNA, 141 bp, synthetic construct
  source 1..141: mol_type = other DNA; note = Hinge 3 encoding sequence
cagcctgacctagctcctggactgaccactattggagcctctcctactcagactgttactctggtgacacaacctgtggttactaaggaaactgccatctccaaactagaaatgccatcttccttgatgttggaggtacct

SEQ ID NO: 21 - DNA, 13176 bp, synthetic construct
  source 1..13176: mol_type = other DNA; note = Vector encoding Microdystrophin 1
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagggtttagtgaaccgtcagatcctgcagaagttggtcgtgaggcactagtccgccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccaagcttgcatgctggggagagatctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagctagcattggacctcgagacgtatcaccgtcacggatactcgactgcacaaagttgctattgactctctttccttatgataattgtgggaattagcaagactataccctccccgtatgcaacgacagaccttacggcaaactggatttatgatatcaacattgcgccgacgagtgatcggggtccaggtagtggagctttcgcagttttatggggaccaccataatgtccgtagaccagcatagttccggtgctttgacctttcgtgagagtctatttgtgtgggaattttgcgtgtttggcgtgttgtctaaactgtgtctttgacgaagatccgcgttgtaccaacataaaggacagtgcggtacagcacgccaactttagcttggaaagtctaaagccagggtagagctctgcgcccccaatcagcttggttatacgaggcgtatcattgaggagtccttatctcccgaatcgcgtggaaattcgcattcgatacaacaaaggcgtgttacttgttgcgtatcggtaggtgttccgcagaagtcctccatgcgctatggccatcaagaaacgaacgtaacgcactggaacattaccaaagtcgcttgtatattccggtcgggtctagacacctagcgtccgactgggttaacctgtcggcccgaaccctcgcaatgtcgtcgtttgcatctatatcagattgtactcaagtacatgtgccaggatatactatattttatgttgatagaagtttgtggaacgcacgatgagaaggagagtatatccccattgggaagctgatgcgacactaccgacagcgacaaccatattcccgcgagctggagtgccttccccaccattggaaatatggaatctgcatcgggtagtttcgtggtaattctgcgctctgtaagccgcacgatgcggttccgggtagtgtgaaaattcgccgcaagtggtaggttcccgatgggccggatgccaatttcggcgttgggggcgtcgcgctggggcgtggggggtgcttacaaaaccaattctatgaaatagccccggctaggcgtgcggagactcattaggtgtcgaattcattgataagcatgaatgctagaatcgcgacaccgctccccacggagctcctctactctaatggcggtacaacgccatgtgctctctcggcaggtacggtcagtgcattcgcaccgtttccgcccattcgcgggtctttcggataaaaaatgaatgctagtagatagcgctaatgtacccctatgatgatctagccataccaaacattcacgtctctttatcagcccccgaatgctttgttcataataccatagctgtcggatcaagagctaatttgggtatggtgtaagctctgtatggcgctccacatttggaaatacaccggtctttgatgacctattcataagtgtaatggcattgttcaaaccgcaaactcctgaaaccagagctacacggacctcataccgtttagagctcatgaaattagacggctgaaatcctgctttggagttcagatcgctcctaaaactatgcaggccgcctgcgtccctagtgctacggagactatgccaatactggtatgaggccactggtattaacgtacaccgtcgttaacccgcttgtgaggaatgggtacatctcgtatgctcttgccacggctgtctattgtctccgatttgtccgaggggcttagtatcgttcaattaagacaccggccggaccttaatgggtatagacagctagggcgcaagtacaccgaccggagtggatcttggttttgacggtacagtgtcttaatctaaccaagccaatccggtctatgcggcacagatcccgagctagtcggttacgcggatcaccgtgcaccgtgaatctctcgcagggcctgctcttccacgtattgtggatttgccgagatggacagctcgatggttaaaccagagcttcggtattggcatgtgatgatgctaatagcagaccccggtagtgcccattctatggcctaggtctgattgctctcgtcagtcatagtgaaggacggaggggcagccaaacatccttacgaatcaaacggtgtgcgagtgcaaatgccgccgacgtttaaaagtatcaatactatagtcgccctgtttactctcgctcttcgtggaaccacagagctattgagctaacttgtcacttaaagagaacactggcggctcaccgggacggccgcaaataataggatgccagcagaatgcgctaggtctgttgatgcaacaaaatctagatcgttatcacgacatatcgctcgtgtgcggcgcaacaagctctgtaaatcccacgacgctggattagacgcgtggtggacaggaacgtcacacgggagcctatattcttctattgcctttcaatccgctcccgcagtactttacaggacaacacaagacaggaagagatcaggacgagctcagccgctgatgtgggtctatttgctagcaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttcgtacgcccgaacgacagaatgatgcaagggccgttgccatatgccttctgctccggggcggccgtggaaaaacttgatcttgaggaaatggagtggtcactattaatcttcgaagaccgtttttttatggtggacccggcacgctcgccgagacatttccgtgcaagccaaataacgcctccccgcttggtccagtagttggctacatgataaagtcgatccgctgtggttatatacaatactctttgcttggctcaagtgtgcaggtcttaccccgcggcaatgggtttgctaaacatttagggtctccctcgcacgactaatccattccatctaattgcgggacgttgcgtgcgccttggaggagcctaaggcagtcactattctcctgaggtataccgtaggccagttcgggttgtatgttaagcttcacattactaggcccgcactcgcgtgtatgttggttttagcctatgtttataaaaatatgaaggcggacgttattctctctctgtgcagctgtgagcatgctttcgccacatcacgccttatctccgtttgccccccggcaggtgcctggtgttttgagccacctcgccgctgtctcagctatcttttcaagcatatagtgcttgagcactaggaaaaccccgtcgcatgagccaaatgttgcaaacctggtacgtggaacttacgagcgtcgggtttccgctgcggcgcccttctagacattatgggatgagctgccaattatagactaatggcgagcgaatgaggaatgattctggatgtactactgtaaacttaaatagtgctgttgattctacgggactcataatacgaaaggcagagtttttggatgaaggacgagccaaagcgtaccacagagatatgcatcgagctttacccgaacttgcttcgatgtaacggttaatcccgaacccaaaaccagtctcgggtcagcgtgaaattcctataggtgctgccaacaggcacatttagtgagcgggtttcttcttttatcttagggcaattcgggagggttagaggcagcggctcaacgggcatgattaaaccattccaatggtccgatccgaacacaacaactgctaaatccgctcgtgaggcgaaaacttacacttgggatcaccgtcgatcctaataccccgatcccccctggatcacattttgatcatgtattcatgaattggccggcggatgtgattggcgacaccctgcagcgcggttaactcttcaggaaattacaatccgccaagtcgagaaccgtgtcattaccacctcgttgacagttaaccttatctagcccatgaaccagtggaccactcccaacatatattcccaccagacgcgccattgaccgtggcccggttcttcagatcatacatgttggcgcacgagatctcgttatagcactacagccatatgcggtaccactgctaaccgcctacgctgtgacgcacccacgtgatgattacaccgcggcccatggccggagttcaggtgtctgattctggtggtctggatacgtagtaggaccagggacatatttttcattgtcctacgcgcgaaactccgagttcaagtatatacagtcaggatagtatggttgaactcttacgagtttgcggcatgggcgagcagattaaatgtagatgtacctaacacgtcagcgggatttaatccttgcacaaaccgacgtgagatattgtattctagggcttgtggcgatcagttatagctcctcgagggcgtggaggaaggtccttccgattacaggggaaggatgccaactcactatgactcctccttatggccgcatgtaggtagggacaccgtctcccgccgactttccggtaaatgctccaacgacgcttgtatacggccgtcaactttaagggcaatcgtatcacaacggtgaaatacaatccttgggaagggttgtatcaatccagccgtatcaaatctctatggttagcgaagagacgtccaataatataaccatacccctgaacctaaatgtgtgaaattgctcctcacacgtttccttgctctcggtggtggtcgaagtagtgtccccgtaaaccggaaatcacggtttgtcgacgacacatgccgcccggaagccgtttgaaagagggaagacgtctttcttgttacgtacagtttaactctacggcagtcccgattgtcctcataagtaaaacgtcaggccagccgtatagcacggcatatgaatcgtgactttactacctaaatgcgagggttggtcccccgtaacccgatggttatagaaagacccgtattgaaagatatctagcactgaggcccgctttgccccaatccactgtcctgtacgataagtactaggagatttcggggggtcataaggtgggtgtctatcaaaatgggggagggcgatccttggtactaaagtcaacgaaggtgtcatgcatgcgtcttcgtaacgacgtcatgtctagtgacatccaattaagccgccttggtctagcggcgtacctcgaacacacgaggttcaatgatcggcccccccaacacttcctcccctgaacccggtctagctacgtcgcataaagaatatcggacccgactgggcgggtccaagaacttaaaggcctgtccgtgggtttctacgttagtctaatcgcctgccagttggcggctcagcgtacggattcattaatgcag

SEQ ID NO: 22 - DNA, 13269 bp, synthetic construct
  source 1..13269: mol_type = other DNA; note = Vector encoding Microdystrophin 2
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagggtttagtgaaccgtcagatcctgcagaagttggtcgtgaggcactagtccgccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccaagcttgcatgctggggagagatctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagctagcattggacctcgagacgtatcaccgtcacggatactcgactgcacaaagttgctattgactctctttccttatgataattgtgggaattagcaagactataccctccccgtatgcaacgacagaccttacggcaaactggatttatgatatcaacattgcgccgacgagtgatcggggtccaggtagtggagctttcgcagttttatggggaccaccataatgtccgtagaccagcatagttccggtgctttgacctttcgtgagagtctatttgtgtgggaattttgcgtgtttggcgtgttgtctaaactgtgtctttgacgaagatccgcgttgtaccaacataaaggacagtgcggtacagcacgccaactttagcttggaaagtctaaagccagggtagagctctgcgcccccaatcagcttggttatacgaggcgtatcattgaggagtccttatctcccgaatcgcgtggaaattcgcattcgatacaacaaaggcgtgttacttgttgcgtatcggtaggtgttccgcagaagtcctccatgcgctatggccatcaagaaacgaacgtaacgcactggaacattaccaaagtcgcttgtatattccggtcgggtctagacacctagcgtccgactgggttaacctgtcggcccgaaccctcgcaatgtcgtcgtttgcatctatatcagattgtactcaagtacatgtgccaggatatactatattttatgttgatagaagtttgtggaacgcacgatgagaaggagagtatatccccattgggaagctgatgcgacactaccgacagcgacaaccatattcccgcgagctggagtgccttccccaccattggaaatatggaatctgcatcgggtagtttcgtggtaattctgcgctctgtaagccgcacgatgcggttccgggtagtgtgaaaattcgccgcaagtggtaggttcccgatgggccggatgccaatttcggcgttgggggcgtcgcgctggggcgtggggggtgcttacaaaaccaattctatgaaatagccccggctaggcgtgcggagactcattaggtgtcgaattcattgataagcatgaatgctagaatcgcgacaccgctccccacggagctcctctactctaatggcggtacaacgccatgtgctctctcggcaggtacggtcagtgcattcgcaccgtttccgcccattcgcgggtctttcggataaaaaatgaatgctagtagatagcgctaatgtacccctatgatgatctagccataccaaacattcacgtctctttatcagcccccgaatgctttgttcataataccatagctgtcggatcaagagctaatttgggtatggtgtaagctctgtatggcgctccacatttggaaatacaccggtctttgatgacctattcataagtgtaatggcattgttcaaaccgcaaactcctgaaaccagagctacacggacctcataccgtttagagctcatgaaattagacggctgaaatcctgctttggagttcagatcgctcctaaaactatgcaggccgcctgcgtccctagtgctacggagactatgccaatactggtatgaggccactggtattaacgtacaccgtcgttaacccgcttgtgaggaatgggtacatctcgtatgctcttgccacggctgtctattgtctccgatttgtccgaggggcttagtatcgttcaattaagacaccggccggaccttaatgggtatagacagctagggcgcaagtacaccgaccggagtggatcttggttttgacggtacagtgtcttaatctaaccaagccaatccggtctatgcggcacagatcccgagctagtcggttacgcggatcaccgtgcaccgtgaatctctcgcagggcctgctcttccacgtattgtggatttgccgagatggacagctcgatggttaaaccagagcttcggtattggcatgtgatgatgctaatagcagaccccggtagtgcccattctatggcctaggtctgattgctctcgtcagtcatagtgaaggacggaggggcagccaaacatccttacgaatcaaacggtgtgcgagtgcaaatgccgccgacgtttaaaagtatcaatactatagtcgccctgtttactctcgctcttcgtggaaccacagagctattgagctaacttgtcacttaaagagaacactggcggctcaccgggacggccgcaaataataggatgccagcagaatgcgctaggtctgttgatgcaacaaaatctagatcgttatcacgacatatcgctcgtgtgcggcgcaacaagctctgtaaatcccacgacgctggattagacgcgtggtggacaggaacgtcacacgggagcctatattcttctattgcctttcaatccgctcccgcagtactttacaggacaacacaagacaggaagagatcaggacgagctcagccgctgatgtgggtctatttgctagcaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttcgtacgcccgaacgacagaatgatgcaagggccgttgccatatgccttctgctccggggcggccgtggaaaaacttgatcttgaggaaatggagtggtcactattaatcttcgaagaccgtttttttatggtggacccggcacgctcgccgagacatttccgtgcaagccaaataacgcctccccgcttggtccagtagttggctacatgataaagtcgatccgctgtggttatatacaatactctttgcttggctcaagtgtgcaggtcttaccccgcggcaatgggtttgctaaacatttagggtctccctcgcacgactaatccattccatctaattgcgggacgttgcgtgcgccttggaggagcctaaggcagtcactattctcctgaggtataccgtaggccagttcgggttgtatgttaagcttcacattactaggcccgcactcgcgtgtatgttggttttagcctatgtttataaaaatatgaaggcggacgttattctctctctgtgcagctgtgagcatgctttcgccacatcacgccttatctccgtttgccccccggcaggtgcctggtgttttgagccacctcgccgctgtctcagctatcttttcaagcatatagtgcttgagcactaggaaaaccccgtcgcatgagccaaatgttgcaaacctggtacgtggaacttacgagcgtcgggtttccgctgcggcgcccttctagacattatgggatgagctgccaattatagactaatggcgagcgaatgaggaatgattctggatgtactactgtaaacttaaatagtgctgttgattctacgggactcataatacgaaaggcagagtttttggatgaaggacgagccaaagcgtaccacagagatatgcatcgagctttacccgaacttgcttcgatgtaacggttaatcccgaacccaaaaccagtctcgggtcagcgtgaaattcctataggtgctgccaacaggcacatttagtgagcgggtttcttcttttatcttagggcaattcgggagggttagaggcagcggctcaacgggcatgattaaaccattccaatggtccgatccgaacacaacaactgctaaatccgctcgtgaggcgaaaacttacacttgggatcaccgtcgatcctaataccccgatcccccctggatcacattttgatcatgtattcatgaattggccggcggatgtgattggcgacaccctgcagcgcggttaactcttcaggaaattacaatccgccaagtcgagaaccgtgtcattaccacctcgttgacagttaaccttatctagcccatgaaccagtggaccactcccaacatatattcccaccagacgcgccattgaccgtggcccggttcttcagatcatacatgttggcgcacgagatctcgttatagcactacagccatatgcggtaccactgctaaccgcctacgctgtgacgcacccacgtgatgattacaccgcggcccatggccggagttcaggtgtctgattctggtggtctggatacgtagtaggaccagggacatatttttcattgtcctacgcgcgaaactccgagttcaagtatatacagtcaggatagtatggttgaactcttacgagtttgcggcatgggcgagcagattaaatgtagatgtacctaacacgtcagcgggatttaatccttgcacaaaccgacgtgagatattgtattctagggcttgtggcgatcagttatagctcctcgagggcgtggaggaaggtccttccgattacaggggaaggatgccaactcactatgactcctccttatggccgcatgtaggtagggacaccgtctcccgccgactttccggtaaatgctccaacgacgcttgtatacggccgtcaactttaagggcaatcgtatcacaacggtgaaatacaatccttgggaagggttgtatcaatccagccgtatcaaatctctatggttagcgaagagacgtccaataatataaccatacccctgaacctaaatgtgtgaaattgctcctcacacgtttccttgctctcggtggtggtcgaagtagtgtccccgtaaaccggaaatcacggtttgtcgacgacacatgccgcccggaagccgtttgaaagagggaagacgtctttcttgttacgtacagtttaactctacggcagtcccgattgtcctcataagtaaaacgtcaggccagccgtatagcacggcatatgaatcgtgactttactacctaaatgcgagggttggtcccccgtaacccgatggttatagaaagacccgtattgaaagatatctagcactgaggcccgctttgccccaatccactgtcctgtacgataagtactaggagatttcggggggtcataaggtgggtgtctatcaaaatgggggagggcgatccttggtactaaagtcaacgaaggtgtcatgcatgcgtcttcgtaacgacgtcatgtctagtgacatccaattaagccgccttggtctagcggcgtacctcgaacacacgaggttcaatgatcggcccccccaacacttcctcccctgaacccggtctagctacgtcgcataaagaatatcggacccgactgggcgggtccaagaacttaaaggcctgtccgtgggtttctacgttagtctaatcgcctgccagttggcggctcagcgtacggattcattaatgcag

SEQ ID NO: 23 - DNA, 13371 bp, synthetic construct
  source 1..13371: mol_type = other DNA; note = Vector encoding Microdystrophin 3
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagggtttagtgaaccgtcagatcctgcagaagttggtcgtgaggcactagtccgccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccaagcttgcatgctggggagagatctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagctagcattggacctcgagacgtatcaccgtcacggatactcgactgcacaaagttgctattgactctctttccttatgataattgtgggaattagcaagactataccctccccgtatgcaacgacagaccttacggcaaactggatttatgatatcaacattgcgccgacgagtgatcggggtccaggtagtggagctttcgcagttttatggggaccaccataatgtccgtagaccagcatagttccggtgctttgacctttcgtgagagtctatttgtgtgggaattttgcgtgtttggcgtgttgtctaaactgtgtctttgacgaagatccgcgttgtaccaacataaaggacagtgcggtacagcacgccaactttagcttggaaagtctaaagccagggtagagctctgcgcccccaatcagcttggttatacgaggcgtatcattgaggagtccttatctcccgaatcgcgtggaaattcgcattcgatacaacaaaggcgtgttacttgttgcgtatcggtaggtgttccgcagaagtcctccatgcgctatggccatcaagaaacgaacgtaacgcactggaacattaccaaagtcgcttgtatattccggtcgggtctagacacctagcgtccgactgggttaacctgtcggcccgaaccctcgcaatgtcgtcgtttgcatctatatcagattgtactcaagtacatgtgccaggatatactatattttatgttgatagaagtttgtggaacgcacgatgagaaggagagtatatccccattgggaagctgatgcgacactaccgacagcgacaaccatattcccgcgagctggagtgccttccccaccattggaaatatggaatctgcatcgggtagtttcgtggtaattctgcgctctgtaagccgcacgatgcggttccgggtagtgtgaaaattcgccgcaagtggtaggttcccgatgggccggatgccaatttcggcgttgggggcgtcgcgctggggcgtggggggtgcttacaaaaccaattctatgaaatagccccggctaggcgtgcggagactcattaggtgtcgaattcattgataagcatgaatgctagaatcgcgacaccgctccccacggagctcctctactctaatggcggtacaacgccatgtgctctctcggcaggtacggtcagtgcattcgcaccgtttccgcccattcgcgggtctttcggataaaaaatgaatgctagtagatagcgctaatgtacccctatgatgatctagccataccaaacattcacgtctctttatcagcccccgaatgctttgttcataataccatagctgtcggatcaagagctaatttgggtatggtgtaagctctgtatggcgctccacatttggaaatacaccggtctttgatgacctattcataagtgtaatggcattgttcaaaccgcaaactcctgaaaccagagctacacggacctcataccgtttagagctcatgaaattagacggctgaaatcctgctttggagttcagatcgctcctaaaactatgcaggccgcctgcgtccctagtgctacggagactatgccaatactggtatgaggccactggtattaacgtacaccgtcgttaacccgcttgtgaggaatgggtacatctcgtatgctcttgccacggctgtctattgtctccgatttgtccgaggggcttagtatcgttcaattaagacaccggccggaccttaatgggtatagacagctagggcgcaagtacaccgaccggagtggatcttggttttgacggtacagtgtcttaatctaaccaagccaatccggtctatgcggcacagatcccgagctagtcggttacgcggatcaccgtgcaccgtgaatctctcgcagggcctgctcttccacgtattgtggatttgccgagatggacagctcgatggttaaaccagagcttcggtattggcatgtgatgatgctaatagcagaccccggtagtgcccattctatggcctaggtctgattgctctcgtcagtcatagtgaaggacggaggggcagccaaacatccttacgaatcaaacggtgtgcgagtgcaaatgccgccgacgtttaaaagtatcaatactatagtcgccctgtttactctcgctcttcgtggaaccacagagctattgagctaacttgtcacttaaagagaacactggcggctcaccgggacggccgcaaataataggatgccagcagaatgcgctaggtctgttgatgcaacaaaatctagatcgttatcacgacatatcgctcgtgtgcggcgcaacaagctctgtaaatcccacgacgctggattagacgcgtggtggacaggaacgtcacacgggagcctatattcttctattgcctttcaatccgctcccgcagtactttacaggacaacacaagacaggaagagatcaggacgagctcagccgctgatgtgggtctatttgctagcaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttcgtacgcccgaacgacagaatgatgcaagggccgttgccatatgccttctgctccggggcggccgtggaaaaacttgatcttgaggaaatggagtggtcactattaatcttcgaagaccgtttttttatggtggacccggcacgctcgccgagacatttccgtgcaagccaaataacgcctccccgcttggtccagtagttggctacatgataaagtcgatccgctgtggttatatacaatactctttgcttggctcaagtgtgcaggtcttaccccgcggcaatgggtttgctaaacatttagggtctccctcgcacgactaatccattccatctaattgcgggacgttgcgtgcgccttggaggagcctaaggcagtcactattctcctgaggtataccgtaggccagttcgggttgtatgttaagcttcacattactaggcccgcactcgcgtgtatgttggttttagcctatgtttataaaaatatgaaggcggacgttattctctctctgtgcagctgtgagcatgctttcgccacatcacgccttatctccgtttgccccccggcaggtgcctggtgttttgagccacctcgccgctgtctcagctatcttttcaagcatatagtgcttgagcactaggaaaaccccgtcgcatgagccaaatgttgcaaacctggtacgtggaacttacgagcgtcgggtttccgctgcggcgcccttctagacattatgggatgagctgccaattatagactaatggcgagcgaatgaggaatgattctggatgtactactgtaaacttaaatagtgctgttgattctacgggactcataatacgaaaggcagagtttttggatgaaggacgagccaaagcgtaccacagagatatgcatcgagctttacccgaacttgcttcgatgtaacggttaatcccgaacccaaaaccagtctcgggtcagcgtgaaattcctataggtgctgccaacaggcacatttagtgagcgggtttcttcttttatcttagggcaattcgggagggttagaggcagcggctcaacgggcatgattaaaccattccaatggtccgatccgaacacaacaactgctaaatccgctcgtgaggcgaaaacttacacttgggatcaccgtcgatcctaataccccgatcccccctggatcacattttgatcatgtattcatgaattggccggcggatgtgattggcgacaccctgcagcgcggttaactcttcaggaaattacaatccgccaagtcgagaaccgtgtcattaccacctcgttgacagttaaccttatctagcccatgaaccagtggaccactcccaacatatattcccaccagacgcgccattgaccgtggcccggttcttcagatcatacatgttggcgcacgagatctcgttatagcactacagccatatgcggtaccactgctaaccgcctacgctgtgacgcacccacgtgatgattacaccgcggcccatggccggagttcaggtgtctgattctggtggtctggatacgtagtaggaccagggacatatttttcattgtcctacgcgcgaaactccgagttcaagtatatacagtcaggatagtatggttgaactcttacgagtttgcggcatgggcgagcagattaaatgtagatgtacctaacacgtcagcgggatttaatccttgcacaaaccgacgtgagatattgtattctagggcttgtggcgatcagttatagctcctcgagggcgtggaggaaggtccttccgattacaggggaaggatgccaactcactatgactcctccttatggccgcatgtaggtagggacaccgtctcccgccgactttccggtaaatgctccaacgacgcttgtatacggccgtcaactttaagggcaatcgtatcacaacggtgaaatacaatccttgggaagggttgtatcaatccagccgtatcaaatctctatggttagcgaagagacgtccaataatataaccatacccctgaacctaaatgtgtgaaattgctcctcacacgtttccttgctctcggtggtggtcgaagtagtgtccccgtaaaccggaaatcacggtttgtcgacgacacatgccgcccggaagccgtttgaaagagggaagacgtctttcttgttacgtacagtttaactctacggcagtcccgattgtcctcataagtaaaacgtcaggccagccgtatagcacggcatatgaatcgtgactttactacctaaatgcgagggttggtcccccgtaacccgatggttatagaaagacccgtattgaaagatatctagcactgaggcccgctttgccccaatccactgtcctgtacgataagtactaggagatttcggggggtcataaggtgggtgtctatcaaaatgggggagggcgatccttggtactaaagtcaacgaaggtgtcatgcatgcgtcttcgtaacgacgtcatgtctagtgacatccaattaagccgccttggtctagcggcgtacctcgaacacacgaggttcaatgatcggcccccccaacacttcctcccctgaacccggtctagctacgtcgcataaagaatatcggacccgactgggcgggtccaagaacttaaaggcctgtccgtgggtttctacgttagtctaatcgcctgccagttggcggctcagcgtacggattcattaatgcag

SEQ ID NO: 24 - DNA, 13530 bp, synthetic construct
  source 1..13530: mol_type = other DNA; note = Vector encoding Microdystrophin 4
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagggtttagtgaaccgtcagatcctgcagaagttggtcgtgaggcactagtccgccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagaccagcctgacctagctcctggactgaccactattggagcctctcctactcagactgttactctggtgacacaacctgtggttactaaggaaactgccatctccaaactagaaatgccatcttccttgatgttggaggtacctacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtaggcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccaagcttgcatgctggggagagatctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagctagcattggacctcgagacgtatcaccgtcacggatactcgactgcacaaagttgctattgactctctttccttatgataattgtgggaattagcaagactataccctccccgtatgcaacgacagaccttacggcaaactggatttatgatatcaacattgcgccgacgagtgatcggggtccaggtagtggagctttcgcagttttatggggaccaccataatgtccgtagaccagcatagttccggtgctttgacctttcgtgagagtctatttgtgtgggaattttgcgtgtttggcgtgttgtctaaactgtgtctttgacgaagatccgcgttgtaccaacataaaggacagtgcggtacagcacgccaactttagcttggaaagtctaaagccagggtagagctctgcgcccccaatcagcttggttatacgaggcgtatcattgaggagtccttatctcccgaatcgcgtggaaattcgcattcgatacaacaaaggcgtgttacttgttgcgtatcggtaggtgttccgcagaagtcctccatgcgctatggccatcaagaaacgaacgtaacgcactggaacattaccaaagtcgcttgtatattccggtcgggtctagacacctagcgtccgactgggttaacctgtcggcccgaaccctcgcaatgtcgtcgtttgcatctatatcagattgtactcaagtacatgtgccaggatatactatattttatgttgatagaagtttgtggaacgcacgatgagaaggagagtatatccccattgggaagctgatgcgacactaccgacagcgacaaccatattcccgcgagctggagtgccttccccaccattggaaatatggaatctgcatcgggtagtttcgtggtaattctgcgctctgtaagccgcacgatgcggttccgggtagtgtgaaaattcgccgcaagtggtaggttcccgatgggccggatgccaatttcggcgttgggggcgtcgcgctggggcgtggggggtgcttacaaaaccaattctatgaaatagccccggctaggcgtgcggagactcattaggtgtcgaattcattgataagcatgaatgctagaatcgcgacaccgctccccacggagctcctctactctaatggcggtacaacgccatgtgctctctcggcaggtacggtcagtgcattcgcaccgtttccgcccattcgcgggtctttcggataaaaaatgaatgctagtagatagcgctaatgtacccctatgatgatctagccataccaaacattcacgtctctttatcagcccccgaatgctttgttcataataccatagctgtcggatcaagagctaatttgggtatggtgtaagctctgtatggcgctccacatttggaaatacaccggtctttgatgacctattcataagtgtaatggcattgttcaaaccgcaaactcctgaaaccagagctacacggacctcataccgtttagagctcatgaaattagacggctgaaatcctgctttggagttcagatcgctcctaaaactatgcaggccgcctgcgtccctagtgctacggagactatgccaatactggtatgaggccactggtattaacgtacaccgtcgttaacccgcttgtgaggaatgggtacatctcgtatgctcttgccacggctgtctattgtctccgatttgtccgaggggcttagtatcgttcaattaagacaccggccggaccttaatgggtatagacagctagggcgcaagtacaccgaccggagtggatcttggttttgacggtacagtgtcttaatctaaccaagccaatccggtctatgcggcacagatcccgagctagtcggttacgcggatcaccgtgcaccgtgaatctctcgcagggcctgctcttccacgtattgtggatttgccgagatggacagctcgatggttaaaccagagcttcggtattggcatgtgatgatgctaatagcagaccccggtagtgcccattctatggcctaggtctgattgctctcgtcagtcatagtgaaggacggaggggcagccaaacatccttacgaatcaaacggtgtgcgagtgcaaatgccgccgacgtttaaaagtatcaatactatagtcgccctgtttactctcgctcttcgtggaaccacagagctattgagctaacttgtcacttaaagagaacactggcggctcaccgggacggccgcaaataataggatgccagcagaatgcgctaggtctgttgatgcaacaaaatctagatcgttatcacgacatatcgctcgtgtgcggcgcaacaagctctgtaaatcccacgacgctggattagacgcgtggtggacaggaacgtcacacgggagcctatattcttctattgcctttcaatccgctcccgcagtactttacaggacaacacaagacaggaagagatcaggacgagctcagccgctgatgtgggtctatttgctagcaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttcgtacgcccgaacgacagaatgatgcaagggccgttgccatatgccttctgctccggggcggccgtggaaaaacttgatcttgaggaaatggagtggtcactattaatcttcgaagaccgtttttttatggtggacccggcacgctcgccgagacatttccgtgcaagccaaataacgcctccccgcttggtccagtagttggctacatgataaagtcgatccgctgtggttatatacaatactctttgcttggctcaagtgtgcaggtcttaccccgcggcaatgggtttgctaaacatttagggtctccctcgcacgactaatccattccatctaattgcgggacgttgcgtgcgccttggaggagcctaaggcagtcactattctcctgaggtataccgtaggccagttcgggttgtatgttaagcttcacattactaggcccgcactcgcgtgtatgttggttttagcctatgtttataaaaatatgaaggcggacgttattctctctctgtgcagctgtgagcatgctttcgccacatcacgccttatctccgtttgccccccggcaggtgcctggtgttttgagccacctcgccgctgtctcagctatcttttcaagcatatagtgcttgagcactaggaaaaccccgtcgcatgagccaaatgttgcaaacctggtacgtggaacttacgagcgtcgggtttccgctgcggcgcccttctagacattatgggatgagctgccaattatagactaatggcgagcgaatgaggaatgattctggatgtactactgtaaacttaaatagtgctgttgattctacgggactcataatacgaaaggcagagtttttggatgaaggacgagccaaagcgtaccacagagatatgcatcgagctttacccgaacttgcttcgatgtaacggttaatcccgaacccaaaaccagtctcgggtcagcgtgaaattcctataggtgctgccaacaggcacatttagtgagcgggtttcttcttttatcttagggcaattcgggagggttagaggcagcggctcaacgggcatgattaaaccattccaatggtccgatccgaacacaacaactgctaaatccgctcgtgaggcgaaaacttacacttgggatcaccgtcgatcctaataccccgatcccccctggatcacattttgatcatgtattcatgaattggccggcggatgtgattggcgacaccctgcagcgcggttaactcttcaggaaattacaatccgccaagtcgagaaccgtgtcattaccacctcgttgacagttaaccttatctagcccatgaaccagtggaccactcccaacatatattcccaccagacgcgccattgaccgtggcccggttcttcagatcatacatgttggcgcacgagatctcgttatagcactacagccatatgcggtaccactgctaaccgcctacgctgtgacgcacccacgtgatgattacaccgcggcccatggccggagttcaggtgtctgattctggtggtctggatacgtagtaggaccagggacatatttttcattgtcctacgcgcgaaactccgagttcaagtatatacagtcaggatagtatggttgaactcttacgagtttgcggcatgggcgagcagattaaatgtagatgtacctaacacgtcagcgggatttaatccttgcacaaaccgacgtgagatattgtattctagggcttgtggcgatcagttatagctcctcgagggcgtggaggaaggtccttccgattacaggggaaggatgccaactcactatgactcctccttatggccgcatgtaggtagggacaccgtctcccgccgactttccggtaaatgctccaacgacgcttgtatacggccgtcaactttaagggcaatcgtatcacaacggtgaaatacaatccttgggaagggttgtatcaatccagccgtatcaaatctctatggttagcgaagagacgtccaataatataaccatacccctgaacctaaatgtgtgaaattgctcctcacacgtttccttgctctcggtggtggtcgaagtagtgtccccgtaaaccggaaatcacggtttgtcgacgacacatgccgcccggaagccgtttgaaagagggaagacgtctttcttgttacgtacagtttaactctacggcagtcccgattgtcctcataagtaaaacgtcaggccagccgtatagcacggcatatgaatcgtgactttactacctaaatgcgagggttggtcccccgtaacccgatggttatagaaagacccgtattgaaagatatctagcactgaggcccgctttgccccaatccactgtcctgtacgataagtactaggagatttcggggggtcataaggtgggtgtctatcaaaatgggggagggcgatccttggtactaaagtcaacgaaggtgtcatgcatgcgtcttcgtaacgacgtcatgtctagtgacatccaattaagccgccttggtctagcggcgtacctcgaacacacgaggttcaatgatcggcccccccaacacttcctcccctgaacccggtctagctacgtcgcataaagaatatcggacccgactgggcgggtccaagaacttaaaggcctgtccgtgggtttctacgttagtctaatcgcctgccagttggcggctcagcgtacggattcattaatgcag

SEQ ID NO: 25 - protein, 1094 aa, synthetic construct
  source 1..1094: note = Microdystrophin 1
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 26 - protein, 1125 aa, synthetic construct
  source 1..1125: note = Microdystrophin 2
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 27 - protein, 1159 aa, synthetic construct
  source 1..1159: note = Microdystrophin 3
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 28 - protein, 1212 aa, synthetic construct
  source 1..1212: note = Microdystrophin 4
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLP

(19) United States
(12) Patent Application Publication
(10) Pub. No.: US 2025/0099620 A1
(43) Pub. Date: Mar. 27, 2025

(54) GENE THERAPY COMPOSITION AND TREATMENT FOR DYSTROPHIN-RELATED CARDIOMYOPATHY

(71) Applicant: DINAQOR AG, Schlieren (CH)

(72) Inventor: Valeria RICOTTI, London (GB)

(21) Appl. No.: 18/729,031

(22) PCT Filed: Jan. 17, 2023
(86) PCT No.: PCT/EP2023/050934

§ 371 Date: Jul. 15, 2024

Related U.S. Application Data
(60) Provisional application No. 63/300,167, filed on Jan. 17, 2022.

(51) Int. Cl. A61K 48/00; A61K 38/17; A61P 9/06; C12N 15/86

(52) U.S. Cl. CPC A61K 48/0058; A61K 38/1719; A61P 9/06; C12N 15/86; C12N 2750/14143

(57) ABSTRACT

Disclosed are a composition and method of treating or preventing cardiomyopathy in a human subject. In one embodiment, a method comprises delivering a gene therapy drug to cardiac tissue of the human subject. The gene therapy drug comprises a vector comprising a polynucleotide coding sequence for a microdystrophin protein.

### CROSS-REFERENCE TO RELATED APPLICATION(S)

This application claims the benefit of priority of U.S. Provisional Patent Application No. 63/300,167, filed on Jan. 17, 2022, the disclosure of which is hereby incorporated by reference herein in its entirety.

### FIELD OF THE INVENTION

The present invention relates to the treatment of cardiac diseases (e.g., cardiac myopathies), and, more specifically, to gene therapy methods and pharmaceutical compositions for the treatment of dystrophin-related cardiomyopathy.

### BACKGROUND OF THE INVENTION

Despite pharmacologic advances in the treatment of various heart conditions, such as heart failure, mortality, and morbidity remain unacceptably high. Furthermore, certain therapeutic approaches are not suitable for many patients (e.g., ones who have an advanced heart failure condition associated with other co-morbid diseases). Alternative approaches, such as gene therapy and cell therapy, have attracted increased attention due to their potential to be uniquely tailored and efficacious in addressing the root cause pathogenesis of many cardiac diseases.

### OBJECTS AND SUMMARY OF THE INVENTION

It is an object of certain embodiments of the present invention to provide methods of delivering therapeutic polynucleotide sequences to cardiomyocytes of a human subject.

It is a further object of certain embodiments of the present invention to utilize gene therapy methods for treating dystrophin-related cardiomyopathy.

It is a further object of certain embodiments of the present invention to vectorize a polynucleotide sequence encoding for a microdystrophin construct.

It is a further object of certain embodiments of the present invention to deliver a vectorized microdystrophin construct to cardiac tissue of a human patient.

The above objects and others are met by the present invention, where certain embodiments are directed to a gene therapy drug for treating or preventing cardiomyopathy in a human subject, the gene therapy drug comprising: a vector comprising a polynucleotide sequence encoding for a microdystrophin protein, wherein the polynucleotide coding sequence encodes for a subset of repeat units R1-R24 of a dystrophin protein, and wherein the repeat units of the subset are selected from a group consisting of R1, R2, R24, portions thereof, and combinations thereof with the proviso that R1 and R24, or portions thereof, are present in the subset.

In at least one embodiment, the polynucleotide coding sequence encodes for only hinge 1, hinge 3, and hinge 4 of the hinge domains of the dystrophin protein.

In at least one embodiment, the polynucleotide coding sequence encodes for only hinge 1 and hinge 4 of the hinge domains of the dystrophin protein.

In at least one embodiment, the repeat units of the subset consist of R1 and R24, or portions thereof. In at least one embodiment, the polynucleotide coding sequence encodes for the entire CT domain of the dystrophin protein. In at least one embodiment, the polynucleotide coding sequence encodes for about 65% to about 85% of R1 and about 20% to about 40% of R24. In at least one embodiment, the polynucleotide coding sequence encodes for about 65% to about 85% of R1 and about 50% to about 70% of R24 with the proviso that the polynucleotide sequence completely encodes exon 60 of the dystrophin gene. In at least one embodiment, the polynucleotide coding sequence encodes for about 95% or greater of R1 and about 60% or less of R24 with the proviso that the polynucleotide sequence completely encodes exon 60 of the dystrophin gene.

In at least one embodiment, the polynucleotide sequence encodes for only exons 70-75, or portions thereof, of the cysteine-rich domain of the dystrophin protein. In at least one embodiment, the polynucleotide sequence further encodes for at least a portion of repeat unit R2. In at least one embodiment, the polynucleotide sequence encodes for only hinge 1, hinge 3, and hinge 4 of the hinge domains of the dystrophin protein, and wherein the repeat units of the subset consist of R1, R2, and R24, or portions thereof.

In at least one embodiment, a gene therapy drug comprising a polynucleotide sequence comprising a microdystrophin-encoding sequence that is at least 90% identical to SEQ ID NO: 2, SEQ ID NO: 3, SEQ ID NO: 4, or SEQ ID NO: 5.

In at least one embodiment, the polynucleotide sequence further encodes a cardiac muscle-specific promoter. In at least one embodiment, the cardiac muscle-specific promoter is a TNNT2 promoter.

In at least one embodiment, the vector comprises a viral vector. In at least one embodiment, the viral vector is an AAV vector. In at least one embodiment, the AAV vector comprises AAV9.

In at least one embodiment, the product of expression of the polynucleotide coding sequence in the human subject is capable of restoring syntrophin binding and localization in dystrophin-deficient heart muscle.

In at least one embodiment, a method of treating or preventing dystrophin-related cardiomyopathy in a human subject comprises: delivering any of the aforementioned gene therapy drugs to cardiac tissue of the human subject. In at least one embodiment, the dystrophin-related cardiomyopathy is associated with Duchenne muscular dystrophy or Becker muscular dystrophy.

At least one embodiment relates to a functional microdystrophin protein encoded by SEQ ID NO: 2.

At least one embodiment relates to a functional microdystrophin protein encoded by SEQ ID NO: 3.

At least one embodiment relates to a functional microdystrophin protein encoded by SEQ ID NO: 4.

At least one embodiment relates to a functional microdystrophin protein encoded by SEQ ID NO: 5.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 illustrates exemplary microdystrophin constructs in accordance with embodiments of the present disclosure;

FIG. 2A is a plot of vector copy numbers analysis in mouse hearts at 4 weeks after delivery of microdystrophin constructs in accordance with embodiments of the present disclosure;

FIG. 2B is a plot of vector copy numbers analysis in mouse hearts at 12 weeks after delivery of microdystrophin constructs in accordance with embodiments of the present disclosure;

FIG. 3 is a plot of vector copy numbers analysis (mean per group) in mouse hearts at 4 weeks and 12 weeks after delivery of microdystrophin constructs in accordance with embodiments of the present disclosure;

FIG. 4 is a plot of relative quantification of the microdystrophin mRNA in the heart tissue of each experimental group;

FIG. 5 is a plot of relative quantification of the microdystrophin mRNA in the heart tissue (mean per group);

FIG. 6A is a Western Blot assay indicating the end of the CT domain for expressed microdystrophin;

FIG. 6B is a Western Blot assay indicating the R1 domain for expressed microdystrophin;

FIG. 7 is an immunofluorescence representation of microdystrophin expression in mdx^{4CV} mouse hearts;

FIG. 8 depicts normal syntrophin expression in normal mouse heart, abnormal syntrophin expression in mdx^{4CV} mouse hearts, and restored syntrophin expression after gene therapy with microdystrophin 1, 2, and 4 in accordance with embodiments of the present disclosure;

FIG. 9A shows plots of heart rate, QRS width, and QT interval derived from electrocardiograph measurements at three months post-injection of microdystrophin constructs in neonatal mice;

FIG. 9B shows plots of heart rate, QRS width, and QT interval derived from electrocardiograph measurements at six months post-injection of microdystrophin constructs in neonatal mice;

FIG. 10A shows structural remodeling of the left ventricle at three months post-injection of microdystrophin constructs in neonatal mice;

FIG. 10B shows structural remodeling of the left ventricle at six months post-injection of microdystrophin constructs in neonatal mice;

FIG. 11A shows plots of systolic function and diastolic function at three months post-injection of microdystrophin constructs in neonatal mice;

FIG. 11B shows plots of systolic function and diastolic function at six months post-injection of microdystrophin constructs in neonatal mice;

FIG. 12 shows plots of biodistribution as measured in different areas of the heart and liver at three months post-injection of microdystrophin constructs in neonatal mice;

FIG. 13 shows western blot images assessing microdystrophin expression in the tissue of the left ventricles at three months post-injection;

FIG. 14 shows expression levels of microdystrophin in the septum end ventricles of the heart;

FIG. 15 shows resulting beating rate and force for treatment after microdystrophin expression in engineered heart tissue;

FIG. 16 shows arrhythmia score after microdystrophin expression in engineered heart tissue;

FIG. 17 illustrates a vector for an exemplary microdystrophin construct according to a first embodiment;

FIG. 18 illustrates a vector for an exemplary microdystrophin construct according to a second embodiment;

FIG. 19 illustrates a vector for an exemplary microdystrophin construct according to a third embodiment; and

FIG. 20 illustrates a vector for an exemplary microdystrophin construct according to a fourth embodiment.

### DEFINITIONS

As used herein, the singular forms “a,” “an,” and “the” include plural references unless the context clearly indicates otherwise. Thus, for example, reference to “a drug” includes a single drug as well as a mixture of two or more different drugs; and reference to a “viral vector” includes a single viral vector as well as a mixture of two or more different viral vectors, and the like.

Also as used herein, “about,” when used in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

Also as used herein, “polynucleotide” has its ordinary and customary meaning in the art and includes any polymeric nucleic acid such as DNA or RNA molecules, as well as chemical derivatives known to those skilled in the art. Polynucleotides include not only those encoding a therapeutic protein, but also include sequences that can be used to decrease the expression of a targeted nucleic acid sequence using techniques known in the art (e.g., antisense, interfering, or small interfering nucleic acids). Polynucleotides can also be used to initiate or increase the expression of a targeted nucleic acid sequence or the production of a targeted protein within cells of the cardiovascular system. Targeted nucleic acids and proteins include, but are not limited to, nucleic acids and proteins normally found in the targeted tissue, derivatives of such naturally occurring nucleic acids or proteins, naturally occurring nucleic acids or proteins not normally found in the targeted tissue, or synthetic nucleic acids or proteins. One or more polynucleotides can be used in combination, administered simultaneously and/or sequentially, to increase and/or decrease one or more targeted nucleic acid sequences or proteins.

Also as used herein, “exogenous” nucleic acids or genes are those that do not occur in nature in the vector utilized for nucleic acid transfer; e.g., not naturally found in the viral vector, but the term is not intended to exclude nucleic acids encoding a protein or polypeptide that occurs naturally in the patient or host.

Also as used herein, “cardiac cell” includes any cell of the heart that is involved in maintaining a structure or providing a function of the heart such as a cardiac muscle cell, a cell of the cardiac vasculature, or a cell present in a cardiac valve. Cardiac cells include cardio myocytes (having both normal and abnormal electrical properties), epithelial cells, endothelial cells, fibroblasts, cells of the conducting tissue, cardiac pace making cells, and neurons.

Also as used herein, “adeno-associated virus” or “AAV” encompasses all subtypes, serotypes and pseudotypes, as well as naturally occurring and recombinant forms. A variety of AAV serotypes and strains are known in the art and are publicly available from sources, such as the ATCC, and academic or commercial sources. Alternatively, sequences from AAV serotypes and strains which are published and/or available from a variety of databases may be synthesized using known techniques.

Also as used herein, “serotype” refers to an AAV which is identified by and distinguished from other AAVs based on capsid protein reactivity with defined antisera. There are at least twelve known serotypes of human AAV, including AAV1 through AAV12, however additional serotypes continue to be discovered, and use of newly discovered serotypes are contemplated.

Also as used herein, “pseudotyped” AAV refers to an AAV that contains capsid proteins from one serotype and a viral genome including 5′ and 3′ inverted terminal repeats (ITRs) of a different or heterologous serotype. A pseudotyped recombinant AAV (rAAV) would be expected to have cell surface binding properties of the capsid serotype and genetic properties consistent with the ITR serotype. A pseudotyped rAAV may comprise AAV capsid proteins, including VP1, VP2, and VP3 capsid proteins, and ITRs from any serotype AAV, including any primate AAV serotype from AAV1 through AAV12, as long as the capsid protein is of a serotype heterologous to the serotype(s) of the ITRs. In a pseudotyped rAAV, the 5′ and 3′ ITRs may be identical or heterologous. Pseudotyped rAAV are produced using standard techniques described in the art.

Also as used herein, “chimeric” rAAV vector encompasses an AAV vector comprising heterologous capsid proteins; that is, a rAAV vector may be chimeric with respect to its capsid proteins VP1, VP2, and VP3, such that VP1, VP2, and VP3 are not all of the same serotype AAV. A chimeric AAV as used herein encompasses AAV wherein the capsid proteins VP1, VP2, and VP3 differ in serotypes, including for example but not limited to capsid proteins from AAV1 and AAV2; are mixtures of other parvo virus capsid proteins or comprise other virus proteins or other proteins, such as for example, proteins that target delivery of the AAV to desired cells or tissues. A chimeric rAAV as used herein also encompasses an rAAV comprising chimeric 5′ and 3′ ITRs.

Also as used herein, a “pharmaceutically acceptable excipient or carrier” refers to any inert ingredient in a composition that is combined with an active agent in a formulation. A pharmaceutically acceptable excipient can include, but is not limited to, carbohydrates (such as glucose, sucrose, or dextrans), antioxidants (such as ascorbic acid or glutathione), chelating agents, low-molecular weight proteins, high-molecular weight polymers, gel-forming agents, or other stabilizers and additives. Other examples of a pharmaceutically acceptable carrier include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

Also as used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

Also as used herein, a “subject” encompasses the definition of the term “patient” and does not exclude individuals who are otherwise healthy.

Also as used herein, “treatment of” and “treating” include the administration of a drug with the intent to lessen the severity of or prevent a condition, e.g., heart disease.

Also as used herein, “prevention of” and “preventing” include the avoidance of the onset of a condition, e.g., heart disease.

Also as used herein, a “condition” or “conditions” refers to those medical conditions, such as heart disease, that can be treated, mitigated, or prevented by administration to a subject of an effective amount of a drug.

Also as used herein, an “effective amount” refers to the amount of a drug that is sufficient to produce a beneficial or desired effect at a level that is readily detectable by a method commonly used for detection of such an effect. In some embodiments, such an effect results in a change of at least 10% from the value of a basal level where the drug is not administered. In other embodiments, the change is at least 20%, 50%, 80%, or an even higher percentage from the basal level. As will be described below, the effective amount of a drug may vary from subject to subject, depending on age, general condition of the subject, the severity of the condition being treated, the particular drug administered, and the like. An appropriate “effective” amount in any individual case may be determined by one of ordinary skill in the art by reference to the pertinent texts and literature and/or by using routine experimentation.

Also as used herein, an “active agent” refers to any material that is intended to produce a therapeutic, prophylactic, or other intended effect, whether or not approved by a government agency for that purpose.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

The present invention relates to microdystrophin constructs for treating dystrophin-related cardiomyopathies. Dystrophin is a cytoplasmic protein that is part of a protein complex that connects the cytoskeleton of muscle fibers to the extracellular matrix via the cell membrane. The DMD gene, which encodes for the dystrophin protein, is about 2.3 megabases in length and is located at the locus Xp21. SEQ ID NO:1 is the 79-exon muscle transcript of the DMD gene, which includes 11,058 base pairs (bp). The full dystrophin protein includes the following regions from the N-terminus to the C-terminus: an actin binding domain, hinge 1 (H1), repeat units 1-3, hinge 2 (H2), repeat units 4-19, hinge 3 (H3), repeat units 20-24, hinge 4 (H4), a cysteine-rich (CR) domain that partially overlaps with H4, and a C-terminus region. Mutations in the dystrophin gene (i.e., spontaneous mutations or inherited mutations) can result in different forms of muscular dystrophy. The most common disorders caused by defects in dystrophin are Duchenne muscular dystrophy and Becker muscular dystrophy.

Microdystrophin gene therapy is an approach that utilizes shorter but functional variants of the dystrophin protein (referred to as “microdystrophin protein” or “microdystrophin”) to reduce muscle damage in muscular dystrophy patients. Embodiments of the present application relate to various microdystrophin constructs that are targeted to treat cardiomyopathy. For example, such constructs may be expressed using a cardiac-specific promoter (e.g., the TNNT promoter). Such constructs can be used for the treatment of Duchenne muscular dystrophy, Becker muscular dystrophy, and other X-linked disorders resulting from abnormal dystrophin. In certain embodiments, the microdystrophin constructs described herein contain full CR domains and full or nearly full C-terminus regions (e.g., up to exon 75, which includes the syntrophin binding domain that is advantageous for binding ion channels in heart muscle).

At present, for all inherited diseases and heart failure, the only curative treatment is heart transplantation. Cardiac gene therapy with AAV-based vectors holds great promise for the treatment of dystrophin-related cardiomyopathies. AAV vectors are non-pathogenic, unable to replicate on their own, persist in the host nucleus in an extra-chromosomal form, and can be delivered by intra-myocardial or intracoronary or systemic injections.

Certain embodiments of the present disclosure relate to different approaches involving a combination of one or more AAVs in connection with AAV-mediated microdystrophin gene expression in cardiomyocytes (e.g., hiPSC-derived cardiomyocytes). In some embodiments, each cassette is packaged into a suitable AAV. For example, the cassettes may each be packaged into rAAV2/9, which is a particularly efficient serotype for cardiomyocyte transduction.

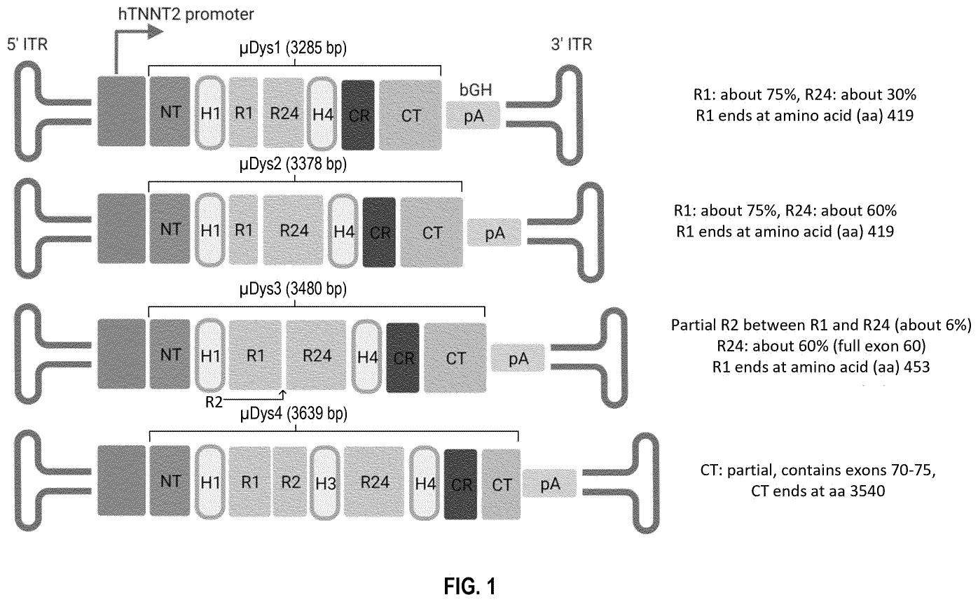

Certain embodiments of the present disclosure relate to expression of functional microdystrophins containing about 25-40% of the total amino acids of the full dystrophin protein (e.g., encoded by nucleic acid sequences having total lengths of 3000 bp to 4000 bp). Exemplary microdystrophin constructs are shown in FIG. 1, which may encode for only a subset of the dystrophin regions (or portions thereof) discussed above. These include:
the N-terminus (NT) region, encoded by SEQ ID NO: 7;
the C-terminus (CT) region, encoded by SEQ ID NO: 11;
the hinge 1 region, encoded by SEQ ID NO: 8;
the hinge 3 region, encoded by SEQ ID NO: 20;
the hinge 4 region, encoded by SEQ ID NO: 9;
the repeat 1 (R1) region, encoded by SEQ ID NO: 13;
the repeat 2 (R2) region, encoded by SEQ ID NO: 19;
the repeat 24 (R24) region, encoded by SEQ ID NO: 17; and
the cysteine-rich (CR) domain, encoded by SEQ ID NO: 10,
   where any given sequence within any microdytrophin construct may be at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to its corresponding sequence above.

In at least one embodiment, one or more of the hinge regions may be present in a microdystrophin. For example, a microdystrophin construct may encode for one or more of hinge 1, hinge 3, or hinge 4 (omitting hinge 2). In at least one embodiment, a microdystrophin construct may encode for hinge 1, hinge 4, and optionally hinge 3.

In at least one embodiment, a microdystrophin construct encoding for the R1 region may encode for less than the entire length of the R1 region, such as less than about 80%, less than about 70%, less than about 60%, or less than about 50% (e.g., about 50% to about 85%, or about 70% to about 80%). An exemplary sequence encoding for a portion of R1 is included as SEQ ID NO: 12.

In at least one embodiment, a microdystrophin construct encoding for the R2 region may encode for less than the entire length of the R2 region, such as less than about 10% (e.g., about 4% to about 8%). An exemplary sequence encoding for a portion of R2 is included as SEQ ID NO: 18.

In at least one embodiment, a microdystrophin construct encoding for the R24 region may encode for less than the entire length of the R24 region, such as less than about 80%, less than about 70%, less than about 60%, less than about 50%, or less than about 40% (e.g., about 50% to about 70%, or about 20% to about 40%). Exemplary sequences encoding for portions of R24 are included as SEQ ID NO: 15 and SEQ ID NO: 16.

In at least one embodiment, a microdystrophin construct encoding for the CT region may encode for less than the entire length of the CT region, such as less than about 70%, less than about 60%, less than about 50%, or less than about 40% (e.g., about 50% to about 60%). An exemplary sequence encoding for a portion of the CT region is included as SEQ ID NO: 14.

In at least one embodiment, the TNNT2 promoter may correspond to a nucleotide sequence encoded by SEQ ID NO: 6.

In at least one embodiment, a microdystrophin construct nucleic acid sequence is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 2. In at least one embodiment, a microdystrophin may have an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 25. For example, exemplary construct 1 (μDys1) of FIG. 1 encodes the following regions of dystrophin: the NT region, hinge 1, about 65-85% of R1, about 20-40% of R24, hinge 4, the CR domain, and the CT region.

In at least one embodiment, a microdystrophin construct nucleic acid sequence is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 3. In at least one embodiment, a microdystrophin may have an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 26. For example, exemplary construct 2 (μDys2) of FIG. 1 encodes the following regions of dystrophin: the NT region, hinge 1, about 65-85% of R1, about 50-70% of R24, hinge 4, the CR domain, and the CT region.

In at least one embodiment, a microdystrophin construct nucleic acid sequence is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 4. In at least one embodiment, a microdystrophin may have an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 27. For example, exemplary construct 3 (μDys3) of FIG. 1 encodes the following regions of dystrophin: the NT region, hinge 1, R1, about 4-10% of R2, about 50-70% of R24 (full exon 60), hinge 4, the CR domain, and the CT region.

In at least one embodiment, a microdystrophin construct nucleic acid sequence is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 5. In at least one embodiment, a microdystrophin may have an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 28. For example, exemplary construct 4 (μDys4) of FIG. 1 encodes the following regions of dystrophin: the NT region, hinge 1, R1, R2, hinge 3, R24, hinge 4, the CR domain, and partially the CT region (exons 70-75).

Although numerous embodiments herein are described with respect to microdystrophin protein, it is to be understood that the expression of additional proteins (e.g., sarcomeric proteins) is contemplated. Additional exemplary proteins include, without limitations, one or more of MYH7, PKP2, SERCA2, MYBPC3, MYL3, MYL2, ACTC1, TPM1, TNNT2, TNNI3, TTN, FHL1, ALPK3, FKRP, utrophin, variants thereof, or combinations thereof. The protein or proteins used may also be functional variants of the proteins mentioned herein and may exhibit a significant amino acid sequence identity compared to the original protein. For instance, the amino acid identity may amount to at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In this context, the term “functional variant” means that the variant of the protein is capable of, partially or completely, fulfilling the function of the naturally occurring corresponding protein. Functional variants of a protein may include, for example, proteins that differ from their naturally occurring counterparts by one or more amino acid substitutions, deletions, or additions.

The amino acid substitutions can be conservative or non-conservative. It is preferred that the substitutions are conservative substitutions, i.e., a substitution of an amino acid residue by an amino acid of similar polarity, which acts as a functional equivalent. Preferably, the amino acid residue used as a substitute is selected from the same group of amino acids as the amino acid residue to be substituted. For example, a hydrophobic residue can be substituted with another hydrophobic residue, or a polar residue can be substituted with another polar residue having the same charge. Functionally homologous amino acids, which may be used for a conservative substitution comprise, for example, non-polar amino acids such as glycine, valine, alanine, isoleucine, leucine, methionine, proline, phenylalanine, and tryptophan. Examples of uncharged polar amino acids comprise serine, threonine, glutamine, asparagine, tyrosine and cysteine. Examples of charged polar (basic) amino acids comprise histidine, arginine, and lysine. Examples of charged polar (acidic) amino acids comprise aspartic acid and glutamic acid.

Also considered as variants are proteins that differ from their naturally occurring counterparts by one or more (e.g., 2, 3, 4, 5, 10, or 15) additional amino acids. These additional amino acids may be present within the amino acid sequence of the original protein (i.e., as an insertion), or they may be added to one or both termini of the protein. Basically, insertions can take place at any position if the addition of amino acids does not impair the capability of the polypeptide to fulfill the function of the naturally occurring protein in the treated subject. Moreover, variants of proteins also comprise proteins in which, compared to the original polypeptide, one or more amino acids are lacking. Such deletions may affect any amino acid position provided that it does not impair the ability to fulfill the normal function of the protein.

Finally, variants of cardiac sarcomeric proteins also refer to proteins that differ from the naturally occurring protein by structural modifications, such as modified amino acids. Modified amino acids are amino acids which have been modified either by natural processes, such as processing or post-translational modifications, or by chemical modification processes known in the art. Typical amino acid modifications comprise phosphorylation, glycosylation, acetylation, O-Linked N-acetylglucosamination, glutathionylation, acylation, branching, ADP ribosylation, crosslinking, disulfide bridge formation, formylation, hydroxylation, carboxylation, methylation, demethylation, amidation, cyclization, and/or covalent or non-covalent bonding to phosphotidylinositol, flavine derivatives, lipoteichonic acids, fatty acids, or lipids.

The therapeutic polynucleotide sequence encoding the target protein may be administered to the subject to be treated in the form of a gene therapy vector, i.e., a nucleic acid construct which comprises the coding sequence, including the translation and termination codons, next to other sequences required for providing expression of the exogenous nucleic acid such as promoters, kozak sequences, polyA signals and the like.

For example, the gene therapy vector may be part of a mammalian expression system. Useful mammalian expression systems and expression constructs are commercially available. Also, several mammalian expression systems are distributed by different manufacturers and can be employed in the present invention, such as plasmid- or viral vector based systems, e.g., LENTI-Smart™ (InvivoGen), GenScript™ Expression vectors, pAdVAntage™ (Promega), ViraPower™ Lentiviral, Adenoviral Expression Systems (Invitrogen), and adeno-associated viral expression systems (Cell Biolabs).

Gene therapy vectors for expressing an exogenous therapeutic polynucleotide sequence of the invention can be, for example, a viral or non-viral expression vector, which is suitable for introducing the exogenous therapeutic polynucleotide sequence into a cell for subsequent expression of the protein encoded by said nucleic acid. The expression vector can be an episomal vector, i.e., one that is capable of self-replicating autonomously within the host cell, or an integrating vector, i.e., one which stably incorporates into the genome of the cell. The expression in the host cell can be constitutive or regulated (e.g., inducible).

In a certain embodiment, the gene therapy vector is a viral expression vector. Viral vectors for use in the present invention may comprise a viral genome in which a portion of the native sequence has been deleted in order to introduce a heterogeneous polynucleotide without destroying the infectivity of the virus. Due to the specific interaction between virus components and host cell receptors, viral vectors are highly suitable for efficient transfer of genes into target cells. Suitable viral vectors for facilitating gene transfer into a mammalian cell can be derived from different types of viruses, for example, from an AAV, an adenovirus, a retrovirus, a herpes simplex virus, a bovine papilloma virus, a lentivirus, a vaccinia virus, a polyoma virus, a sendai virus, orthomyxovirus, paramyxovirus, papovavirus, picornavirus, pox virus, alphavirus, or any other viral shuttle suitable for gene therapy, variations thereof, and combinations thereof.

“Adenovirus expression vector” or “adenovirus” is meant to include those constructs containing adenovirus sequences sufficient (a) to support packaging of the therapeutic polynucleotide sequence construct, and/or (b) to ultimately express a tissue and/or cell-specific construct that has been cloned therein. In one embodiment of the invention, the expression vector comprises a genetically engineered form of adenovirus. Knowledge of the genetic organization of adenovirus, a 36 kilobase (kb), linear, double-stranded DNA virus, allows substitution of large pieces of adenoviral DNA with foreign sequences up to 7 kb.

Adenovirus growth and manipulation is known to those of skill in the art, and exhibits broad host range in vitro and in vivo. This group of viruses can be obtained in high titers, e.g., 10⁹ to 10¹¹ plaque-forming units per mL, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and/or therapeutic potential as in vivo gene transfer vectors.

Retroviruses (also referred to as “retroviral vector”) may be chosen as gene delivery vectors due to their ability to integrate their genes into the host genome, transferring a large amount of foreign genetic material, infecting a broad spectrum of species and cell types and for being packaged in special cell-lines.

The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

The retrovirus can be derived from any of the subfamilies. For example, vectors from Murine Sarcoma Virus, Bovine Leukemia, Virus Rous Sarcoma Virus, Murine Leukemia Virus, Mink-Cell Focus-Inducing Virus, Reticuloendotheliosis Virus, or Avian Leukosis Virus can be used. The skilled person will be able to combine portions derived from different retroviruses, such as LTRs, tRNA binding sites, and packaging signals to provide a recombinant retrovirus. These retroviruses are then normally used for producing transduction competent retroviral vector particles. For this purpose, the vectors are introduced into suitable packaging cell lines. Retroviruses can also be constructed for site-specific integration into the DNA of the host cell by incorporating a chimeric integrase enzyme into the retroviral particle.

Because herpes simplex virus (HSV) is neurotropic, it has generated considerable interest in treating nervous system disorders. Moreover, the ability of HSV to establish latent infections in non-dividing neuronal cells without integrating into the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient multiplicity of infection (MOI) and in a lessened need for repeat dosing. Avirulent variants of HSV have been developed and are readily available for use in gene therapy contexts.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu, and nef are deleted making the vector biologically safe.

Lentiviral vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome. Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus results in a level of expression that is higher than that of conventional vectors, with the transgenes accumulating at 10% or more of the infected cell's protein in 24 hours.

The empty capsids of papovaviruses, such as the mouse polyoma virus, have received attention as possible vectors for gene transfer. The use of empty polyoma was first described when polyoma DNA and purified empty capsids were incubated in a cell-free system. The DNA of the new particle was protected from the action of pancreatic DNase. The reconstituted particles were used for transferring a transforming polyoma DNA fragment to rat FIII cells. The empty capsids and reconstituted particles consist of all three of the polyoma capsid antigens VP1, VP2 and VP3.

AAVs are parvoviruses belonging to the genus Dependovirus. They are small, nonenveloped, single-stranded DNA viruses which require a helper virus in order to replicate. Co-infection with a helper virus (e.g., adenovirus, herpes virus, or vaccinia virus) is necessary in order to form functionally complete AAV virions. In vitro, in the absence of co-infection with a helper virus, AAV establishes a latent state in which the viral genome exists in an episomal form, but infectious virions are not produced. Subsequent infection by a helper virus “rescues” the genome, allowing it to be replicated and packaged into viral capsids, thereby reconstituting the infectious virion. Recent data indicate that in vivo both wild type AAV and recombinant AAV predominantly exist as large episomal concatemers. In one embodiment, the gene therapy vector used herein is an AAV vector. The AAV vector may be purified, replication incompetent, pseudotyped rAAV particles.

AAV are not associated with any known human diseases, are generally not considered pathogenic, and do not appear to alter the physiological properties of the host cell upon integration. AAV can infect a wide range of host cells, including non-dividing cells, and can infect cells from different species. In contrast to some vectors, which are quickly cleared or inactivated by both cellular and humoral responses, AAV vectors have been shown to induce persistent transgene expression in various tissues in vivo. The persistence of recombinant AAV-mediated transgenes in non-diving cells in vivo may be attributed to the lack of native AAV viral genes and the vector's ITR-linked ability to form episomal concatemers.

AAV is an attractive vector system for use in the cell transduction of the present invention as it has a high frequency of persistence as an episomal concatemer and it can infect non-dividing cells, including cardiomyocytes, thus making it useful for delivery of genes into mammalian cells, for example, in tissue culture and in vivo.

Typically, rAAV is made by cotransfecting a plasmid containing the gene of interest flanked by the two AAV terminal repeats and/or an expression plasmid containing the wild-type AAV coding sequences without the terminal repeats, for example pIM45. The cells are also infected and/or transfected with adenovirus and/or plasmids carrying the adenovirus genes required for AAV helper function. Stocks of rAAV made in such fashion are contaminated with adenovirus, which must be physically separated from the rAAV particles (for example, by cesium chloride density centrifugation or column chromatography). Alternatively, adenovirus vectors containing the AAV coding regions and/or cell lines containing the AAV coding regions and/or some or all of the adenovirus helper genes could be used. Cell lines carrying the rAAV DNA as an integrated provirus can also be used.

Multiple serotypes of AAV exist in nature, with at least twelve serotypes (AAV1-AAV12). Despite the high degree of homology, the different serotypes have tropisms for different tissues. Upon transfection, AAV elicits only a minor immune reaction (if any) in the host. Therefore, AAV is highly suited for gene therapy approaches.

The present disclosure may be directed in some embodiments to a drug comprising an AAV vector that is one or more of AAV1, AAV2, AAV3, AAV4, AAV5, AAV6, AAV7, AAV8, AAV9, AAV10, AAV11, AAV12, ANC AAV, chimeric AAV derived thereof, variations thereof, and combinations thereof, which will be even better suitable for high efficiency transduction in the tissue of interest. In certain embodiments, the gene therapy vector is an AAV serotype 1 vector. In certain embodiments, the gene therapy vector is an AAV serotype 2 vector. In certain embodiments, the gene therapy vector is an AAV serotype 3 vector. In certain embodiments, the gene therapy vector is an AAV serotype 4 vector. In certain embodiments, the gene therapy vector is an AAV serotype 5 vector. In certain embodiments, the gene therapy vector is an AAV serotype 6 vector. In certain embodiments, the gene therapy vector is an AAV serotype 7 vector. In certain embodiments, the gene therapy vector is an AAV serotype 8 vector. In certain embodiments, the gene therapy vector is an AAV serotype 9 vector. In certain embodiments, the gene therapy vector is an AAV serotype 10 vector. In certain embodiments, the gene therapy vector is an AAV serotype 11 vector. In certain embodiments, the gene therapy vector is an AAV serotype 12 vector.

In some embodiments, the gene therapy vector may be an AAV serotype having one or more capsid proteins disclosed in U.S. Pat. Nos. 7,198,951 and 7,906,111, the disclosures of which are hereby incorporated by reference herein in their entireties.

A suitable dose of AAV for humans may be in the range of about 1×10⁸ vector genomes per kilogram of body weight (vg/kg) to about 3×10¹⁴ vg/kg, about 1×10⁸ vg/kg, about 1×10⁹ vg/kg, about 1×10¹⁰ vg/kg, about 1×10¹¹ vg/kg, about 1×10¹² vg/kg, about 1×10¹³ vg/kg, or about 1×10¹⁴ vg/kg. The total amount of viral particles or DRP is, is about, is at least, is at least about, is not more than, or is not more than about, 5×10¹⁵ vg/kg, 4×10¹⁵ vg/kg, 3×10¹⁵ vg/kg, 2×10¹⁵ vg/kg, 1×10¹⁵ vg/kg, 9×10¹⁴ vg/kg, 8×10¹⁴ vg/kg, 7×10¹⁴ vg/kg, 6×10¹⁴ vg/kg, 5×10¹⁴ vg/kg, 4×10¹⁴ vg/kg, 3×10¹⁴ vg/kg, 2×10¹⁴ vg/kg, 1×10¹⁴ vg/kg, 9×10¹³ vg/kg, 8×10¹³ vg/kg, 7×10¹³ vg/kg, 6×10¹³ vg/kg, 5×10¹³ vg/kg, 4×10¹³ vg/kg, 3×10¹³ vg/kg, 2×10¹³ vg/kg, 1×10¹³ vg/kg, 9×10¹² vg/kg, 8×10¹² vg/kg, 7×10¹² vg/kg, 6×10¹² vg/kg, 5×10¹² vg/kg, 4×10¹² vg/kg, 3×10¹² vg/kg, 2×10¹² vg/kg, 1×10¹² vg/kg, 9×10¹¹ vg/kg, 8×10¹¹ vg/kg, 7×10¹¹ vg/kg, 6×10¹¹ vg/kg, 5×10¹¹ vg/kg, 4×10¹¹ vg/kg, 3×10¹¹ vg/kg, 2×10¹¹ vg/kg, 1×10¹¹ vg/kg, 9×10¹⁰ vg/kg, 8×10¹⁰ vg/kg, 7×10¹⁰ vg/kg, 6×10¹⁰ vg/kg, 5×10¹⁰ vg/kg, 4×10¹⁰ vg/kg, 3×10¹⁰ vg/kg, 2×10¹⁰ vg/kg, 1×101° vg/kg, 9×10⁹ vg/kg, 8×10⁹ vg/kg, 7×10⁹ vg/kg, 6×10⁹ vg/kg, 5×10⁹ vg/kg, 4×10⁹ vg/kg, 3×10⁹ vg/kg, 2×10⁹ vg/kg, 1×10⁹ vg/kg, 9×10⁸ vg/kg, 8×10⁸ vg/kg, 7×10⁸ vg/kg, 6×10⁸ vg/kg, 5×10⁸ vg/kg, 4×10⁸ vg/kg, 3×10⁸ vg/kg, 2×10⁸ vg/kg, or 1×10⁸ vg/kg, or falls within a range defined by any two of these values. The above listed dosages being in vg/kg heart tissue units.

Apart from viral vectors, non-viral expression constructs may also be used for introducing a gene encoding a target protein or a functioning variant or fragment thereof into a cell of a patient. Non-viral expression vectors which permit the in vivo expression of protein in the target cell include, for example, a plasmid, a modified RNA, a cDNA, antisense oligomers, DNA-lipid complexes, nanoparticles, exosomes, any other non-viral shuttle suitable for gene therapy, variations thereof, and a combination thereof.

Apart from viral vectors and non-viral expression vectors, nuclease systems may also be used, in conjunction with a vector and/or an electroporation system, to enter into a cell of a patient and introduce therein a gene encoding a target protein or a functioning variant or fragment thereof. Exemplary nuclease systems may include, without limitations, a clustered regularly interspaced short palindromic repeats (CRISPR), a DNA cutting enzyme (e.g., Cas9), meganucleases, TALENs, zinc finger nucleases, any other nuclease system suitable for gene therapy, variations thereof, and a combination thereof. For instance, in one embodiment, one viral vector (e.g., AAV) may be used for a nuclease (e.g., CRISPR) and another viral vector (e.g., AAV) may be used for a DNA cutting enzyme (e.g., Cas9) to introduce both (the nuclease and the DNA cutting enzyme) into a target cell.

Other vector delivery systems which can be employed to deliver a therapeutic polynucleotide sequence encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific. Receptor-mediated gene targeting vehicles may include two components: a cell receptor-specific ligand and a DNA-binding agent.

Suitable methods for the transfer of non-viral vectors into target cells are, for example, the lipofection method, the calcium-phosphate co-precipitation method, the DEAE-dextran method and direct DNA introduction methods using micro-glass tubes, ultrasound, electroporation, and the like. Prior to the introduction of the vector, the cardiac muscle cells may be treated with a permeabilization agent, such as phosphatidylcholine, streptolysins, sodium caprate, decanoylcarnitine, tartaric acid, lysolecithin, Triton X-100, and the like. Exosomes may also be used to transfer naked DNA or AAV-encapsidated DNA.

A gene therapy vector of the invention may comprise a promoter that is functionally linked to the nucleic acid sequence encoding to the target protein. The promoter sequence must be compact and ensure a strong expression. Preferably, the promoter provides for an expression of the target protein in the myocardium of the patient that has been treated with the gene therapy vector. In some embodiment, the gene therapy vector comprises a cardiac-specific promoter that is operably linked to the nucleic acid sequence encoding the target protein. As used herein, a “cardiac-specific promoter” refers to a promoter whose activity in cardiac cells is at least 2-fold higher than in any other non-cardiac cell type. Preferably, a cardiac-specific promoter suitable for being used in the vector of the invention has an activity in cardiac cells which is at least 5-fold, at least 10-fold, at least 15-fold, at least 20-fold, at least 25-fold, or at least 50-fold higher compared to its activity in a non-cardiac cell type.

The cardiac-specific promoter may be a selected human promoter, or a promoter comprising a functionally equivalent sequence having at least about 80%, at least about 90%, at least about 95%, at least about 96%, at least about 97%, at least about 98%, or at least about 99% sequence identity to the selected human promoter. An exemplary non-limiting promoter that may be used is a cardiac troponin T promoter (TNNT2). Other non-limiting examples of promoters include the alpha myosin heavy chain promoter, the myosin light chain 2v promoter, the alpha myosin heavy chain promoter, the alpha-cardiac actin promoter, the alpha-tropomyosin promoter, the cardiac troponin C promoter, the cardiac troponin I promoter, the cardiac myosin-binding protein C promoter, and the sarco/endoplasmic reticulum Ca²⁺-ATPase (SERCA) promoter (e.g., isoform 2 of this promoter (SERCA2)).

The vectors useful in the present invention may have varying transduction efficiencies. As a result, the viral or non-viral vector transduces more than, equal to, or at least about 10%, about 20%, about 30%, about 40%, about 50%, about 55%, about 60%, about 65%, about 70%, about 75%, about 80%, about 85%, about 90%, about 95%, about 99%, or 100% of the cells of the targeted vascular territory. More than one vector (viral or non-viral, or combinations thereof) can be used simultaneously or in sequence. This can be used to transfer more than one polynucleotide, and/or target more than one type of cell. Where multiple vectors or multiple agents are used, more than one transduction/transfection efficiency can result.

Pharmaceutical compositions that contain gene therapy vectors may be prepared either as liquid solutions or suspensions. The pharmaceutical composition of the invention can include commonly used pharmaceutically acceptable excipients, such as diluents and carriers. In particular, the composition comprises a pharmaceutically acceptable carrier, e.g., water, saline, Ringer's solution, or dextrose solution. In addition to the carrier, the pharmaceutical composition may also contain emulsifying agents, pH buffering agents, stabilizers, dyes and the like.

In certain embodiments, a pharmaceutical composition will comprise a therapeutically effective gene dose, which is a dose that is capable of preventing or treating cardiomyopathy in a subject, without being toxic to the subject. Prevention or treatment of cardiomyopathy may be assessed as a change in a phenotypic characteristic associated with cardiomyopathy with such change being effective to prevent or treat cardiomyopathy. Thus, a therapeutically effective gene dose is typically one that, when administered in a physiologically tolerable composition, is sufficient to improve or prevent the pathogenic heart phenotype in the treated subject.

In certain embodiments, gene therapy vectors may be transduced into a subject through several different methods, including intravenous delivery, intraarterial delivery, or intraperitoneal delivery. In some embodiments, a gene therapy vector may be administered directly to heart tissue, for example, by intracoronary administration. In some embodiments, tissue transduction of the myocardium may be achieved by catheter-mediated intramyocardial delivery, or by retrograde venous injection into cardiac veins via the coronary sinus, which may be used to transfer vector-free cDNA coupled to or uncoupled to transduction-enhancing carriers into myocardium.

In certain embodiments, the drug will comprise a therapeutically effective gene dose. A therapeutically effective gene dose is one that is capable of preventing or treating a particular heart condition in a patient, without being toxic to the patient.

Heart conditions that may be treated by the methods disclosed herein may include, without limitations, one or more of a genetically determined heart disease (e.g., genetically determined cardiomyopathy), arrhythmic heart disease, heart failure, ischemia, arrhythmia, myocardial infarction, congestive heart failure, transplant rejection, abnormal heart contractility, non-ischemic cardiomyopathy, mitral valve regurgitation, aortic stenosis or regurgitation, abnormal Ca²⁺ metabolism, congenital heart disease, primary or secondary cardiac tumors, and combinations thereof.

### ILLUSTRATIVE EXAMPLES

The following example is set forth to assist in understanding the disclosure and should not, of course, be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein.

Example 1 describes four different microdystrophin constructs utilized in the various studies that follow.

Examples 2-6 describe 4- and 12-week tolerability, gene expression, and protein expression studies following intravenous injection of AAV9-hTNNT-microdystrophin vectors in an mdx^{4CV} mouse model.

Example 7 describes a gene therapy efficacy study was performed on DMD^{mdx} neonatal rats.

Example 8 describes AAV9-microdystrophin transduction in engineered heart tissue.

Each vector used in the studies included a microdystrophin construct having a sequence length below the ˜4.7 kb packaging limit of AAV.

### Example 1: Microdystrophin Constructs

Four different rAAV9 vectors encoding human microdystrophin under the control of the hTNNT2 promoter were prepared for IV injection into mdx^{4CV} mice. FIG. 1 illustrates the microdystrophin constructs used in the Examples. Each vector includes a construct encoding a microdystrophin protein (NT through CT), and further includes a hTNNT2 promoter sequence (SEQ ID NO: 6) and a bovine growth hormone polyadenylation (bGH PolyA) encoding sequence after each CT.

Each construct includes an NT domain-encoding sequence (SEQ ID NO: 7), and a H1-encoding sequence (SEQ ID NO: 8).

Each construct sequence is further flanked by inverted terminal repeat (ITR) sequences.

Construct 1 (μDys1, SEQ ID NO: 2) further includes sequences encoding about 75% of R1 up to amino acid (aa) 419 (SEQ ID NO: 12), about 30% of R24 (SEQ ID NO: 15), H4 (SEQ ID NO: 9), the CR domain (SEQ ID NO: 10), and the CT domain (SEQ ID NO: 11).

Construct 2 (μDys2, SEQ ID NO: 3) further includes sequences encoding about 75% of R1 up to amino acid (aa) 419 (SEQ ID NO: 12), about 60% of R24 including full exon 60 (SEQ ID NO: 16), H4 (SEQ ID NO: 9), the CR domain (SEQ ID NO: 10), and the CT domain (SEQ ID NO: 11).

Construct 3 (μDys3, SEQ ID NO: 4) further includes sequences encoding R1 (SEQ ID NO: 13), an R2 connector sequence (SEQ ID NO: 18), about 60% of R24 including full exon 60 (SEQ ID NO: 16), H4 (SEQ ID NO: 9), the CR domain (SEQ ID NO: 10), and the CT domain (SEQ ID NO: 11).

Construct 4 (μDys4, SEQ ID NO: 5) further includes sequences encoding R1 (SEQ ID NO: 13), R2 (SEQ ID NO: 19), H3 (SEQ ID NO: 20), R24 (SEQ ID NO: 17), H4 (SEQ ID NO: 9), the CR domain (SEQ ID NO: 10), and the CT domain containing only exons 70-75 ending at aa 3540 (SEQ ID NO: 14).

Construct 5 (rAAV9-MCKE-DMD-PC) was used as a positive control.

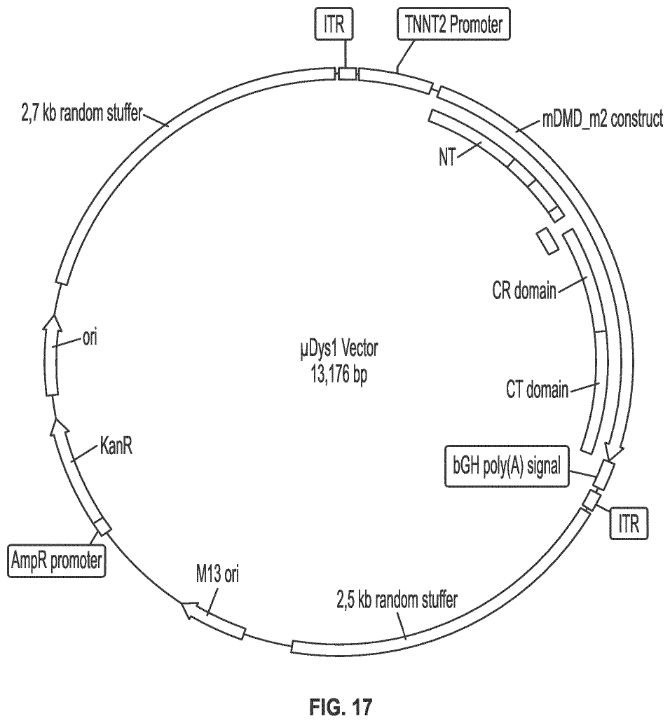

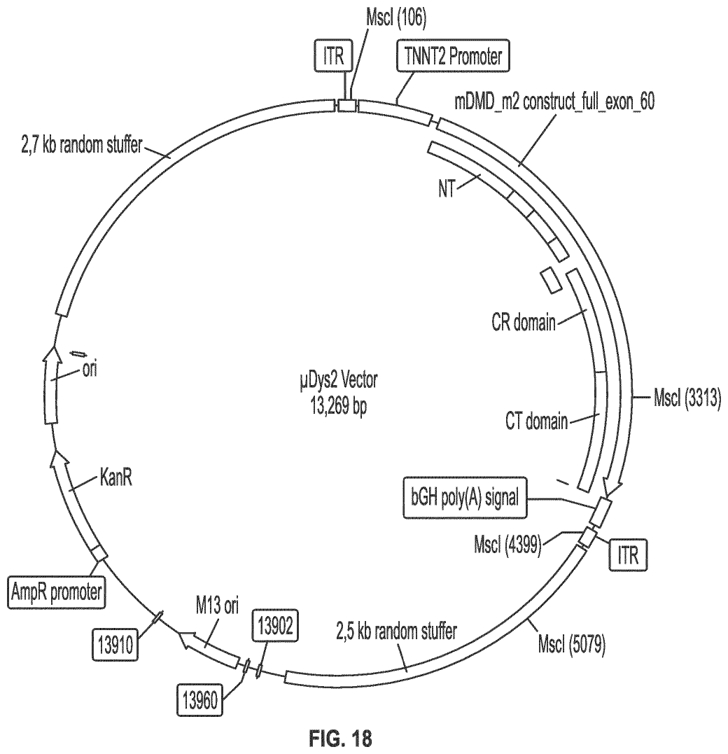

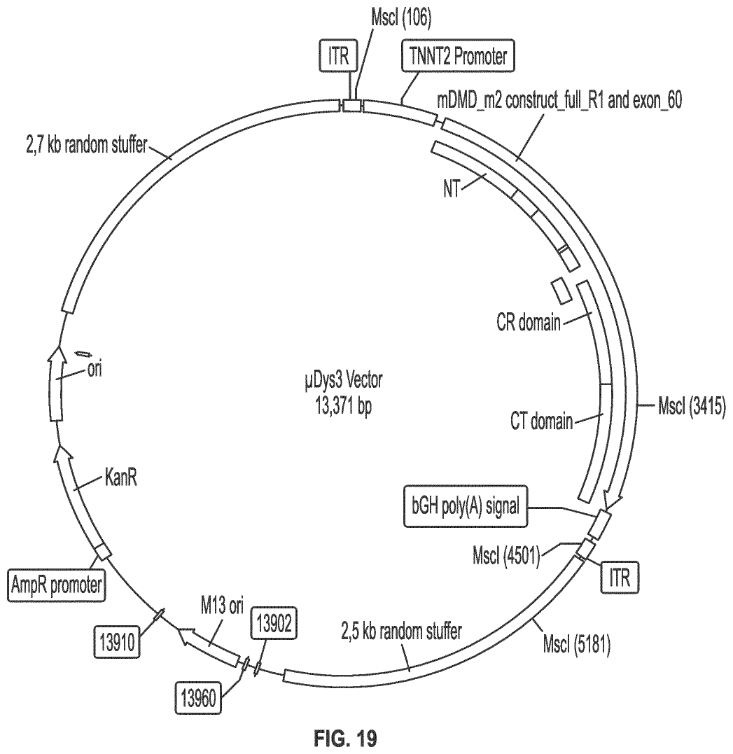

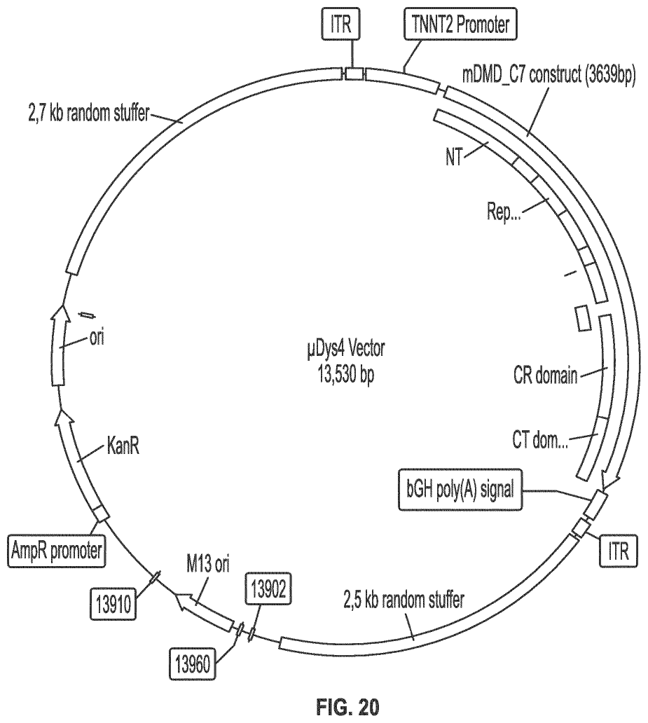

Exemplary AAV-vector sequences incorporating the various microdystrophin constructs and used in the studies are as follows: SEQ ID NO: 21 (FIG. 17); SEQ ID NO: 22 (FIG. 18); SEQ ID NO: 23 (FIG. 19); and SEQ ID NO: 24 (FIG. 20).

### Example 2: Experimental Design

Animals were injected between 11 and 14 weeks of age, and were randomly assigned to the different cohorts. The experimental parameters for each animal group are summarized below in Table 1.

TABLE 1
Experimental Parameters for Treatment with
Microdystrophin Constructs and Controls

| | | | | No. of |
|---|---|---|---|---|
| Group | Treatment | Dose | Duration of Follow-up p.i. | Animals |
| 1A | μDys1 | 10¹⁴ vg/kg | ~4 weeks (28 days ± 3 days) | 5 |
| 2A | μDys2 | 10¹⁴ vg/kg | ~4 weeks (28 days ± 3 days) | 5 |
| 3A | μDys3 | 10¹⁴ vg/kg | ~4 weeks (28 days ± 3 days) | 5 |
| 4A | μDys4 | 10¹⁴ vg/kg | ~4 weeks (28 days ± 3 days) | 5 |
| PC | Positive | 10¹⁴ vg/kg | ~4 weeks (28 days ± 3 days) | 2 |
| | control | | | |
| Veh | Vehicle | N/A | ~4 weeks (28 days ± 3 days) | 5 |
| | control | | | |
| 1B | μDys1 | 10¹⁴ vg/kg | ~12 weeks (84 days ± 6 days) | 5 |
| 2B | μDys2 | 10¹⁴ vg/kg | ~12 weeks (84 days ± 6 days) | 5 |
| 3B | μDys3 | 10¹⁴ vg/kg | ~12 weeks (84 days ± 6 days) | 5 |
| 4B | μDys4 | 10¹⁴ vg/kg | ~12 weeks (84 days ± 6 days) | 5 |
| PC | Positive | 10¹⁴ vg/kg | ~12 weeks (84 days ± 6 days) | 2 |
| | control | | | |
| Veh | Vehicle | N/A | ~12 weeks (84 days ± 6 days) | 5 |
| | control | | | |

### Example 3: Vector Quantification

Vector quantification, including vector copy numbers analysis and vector biodistribution analysis in the heart, was performed using qPCR on gDNA from heart issue (one sample per mouse using the upper half of the heart with left and right ventricles).

gDNA extraction was performed using a Gentra Puregene kit and Tissue Lyser II (both from Qiagen).

Simplex qPCR analyses were performed on 50 ng of gDNA in duplicate on: the microdystrophin sequence (target sequence, using the same assay for all the constructs and targeting the 5′ portion of the CR domain); and the mAlbumin gene (murine endogenous gene).

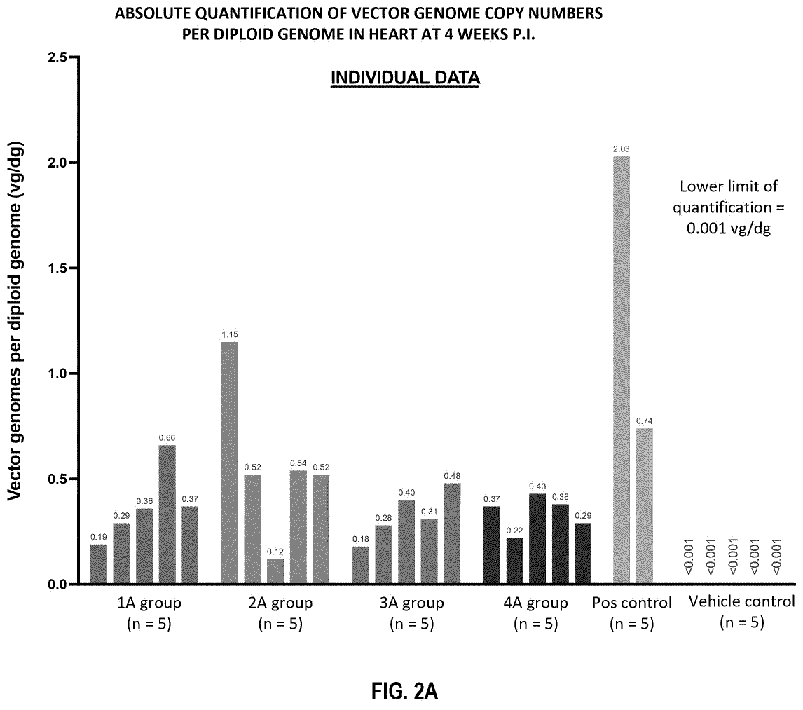

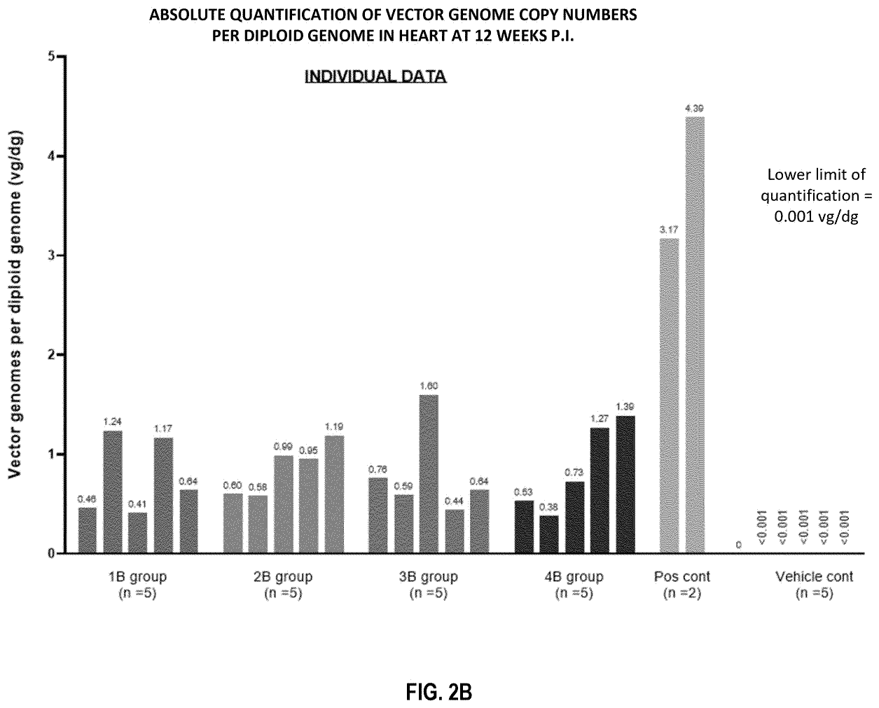

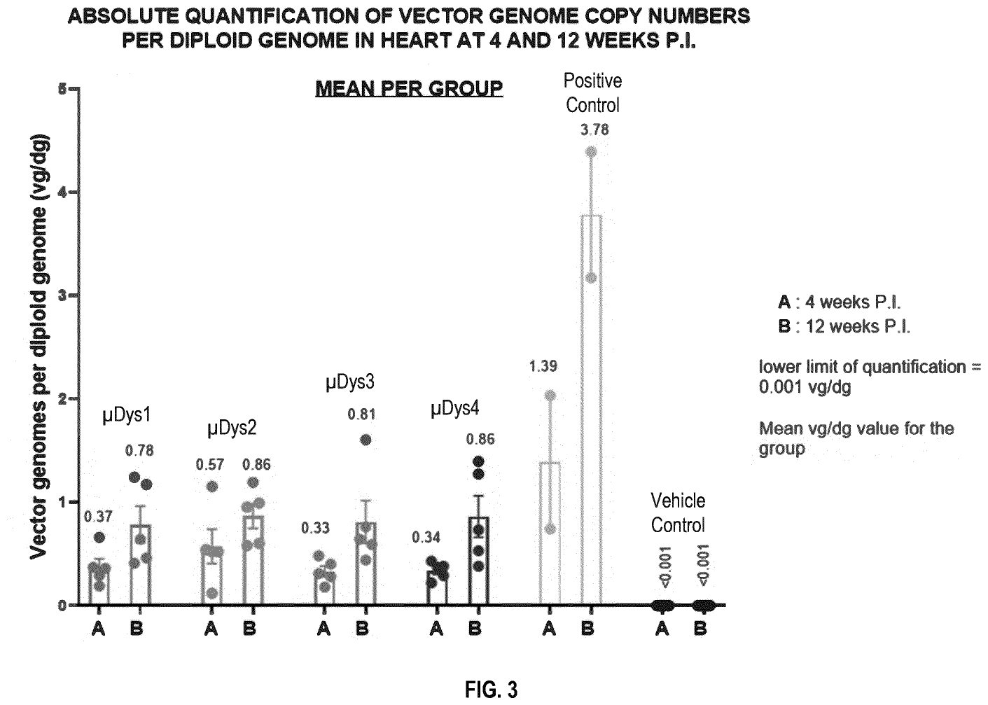

FIGS. 2A and 2B show vector copy number analysis at 4 weeks and 12 weeks, respectively, and FIG. 3 shows this data based on mean per group. This data demonstrates biodistribution of Constructs 1-4 that was comparable to Construct 5 (positive control). Data is expressed as vector genome copies per diploid genome (vg/dg).

### Example 4: Quantification of mRNA

Quantification of microdystrophin mRNA was performed using qPCR on cDNA from heart issue (one sample per mouse using the upper half of the heart with left and right ventricles). Initially, total RNA extraction from the heart tissue was performed using Trizol. DNase I treatment was performed using ezDNase on 500 ng of total RNA. Reverse transcription was performed using a SuperScript IV VILO Master mix on 500 ng of DNase I-treated RNA, followed by dilution (1/40) to obtain cDNA. Simplex Sybr qPCR analyses were performed in duplicate on: the microdystrophin sequence (target sequence); and the mHPRT1 gene (murine endogenous gene).

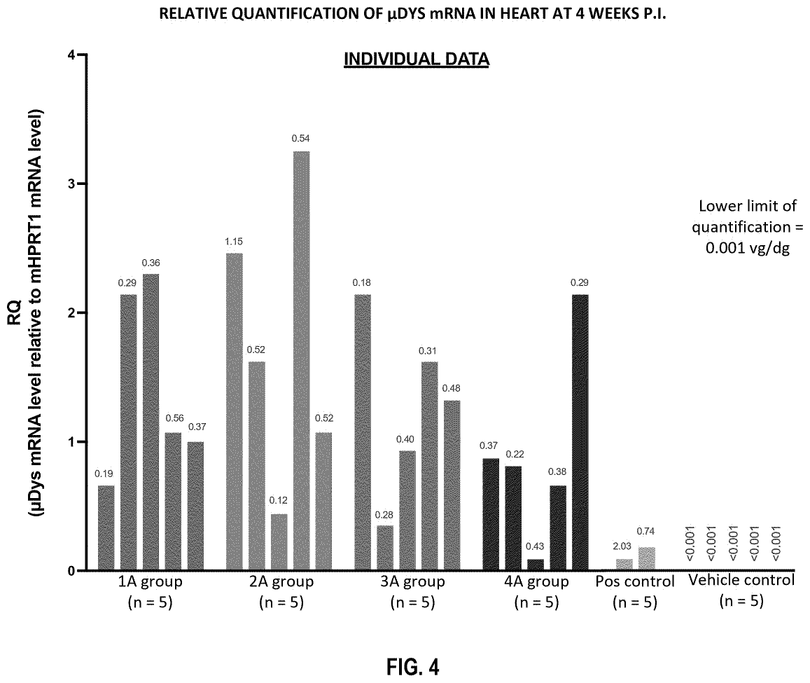

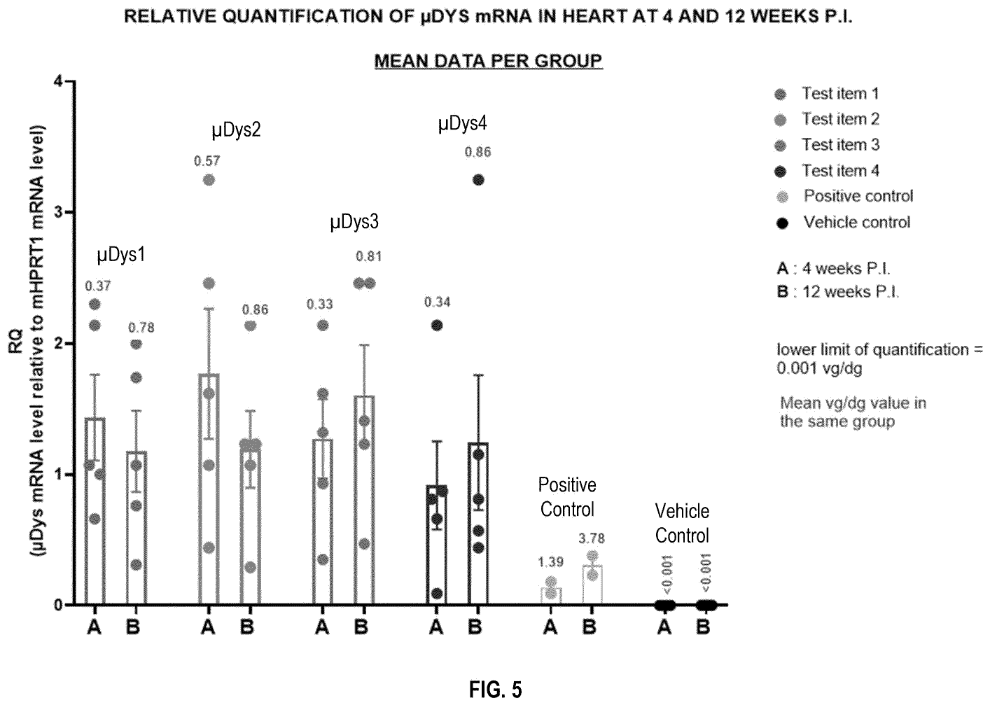

FIGS. 4 and 5 show relative quantification of the microdystrophin mRNA in the heart tissue for individuals and mean per group, respectively, demonstrating increased expression of Constructs 1-4 over positive control (Construct 5).

### Example 5: Microdystrophin Protein Expression

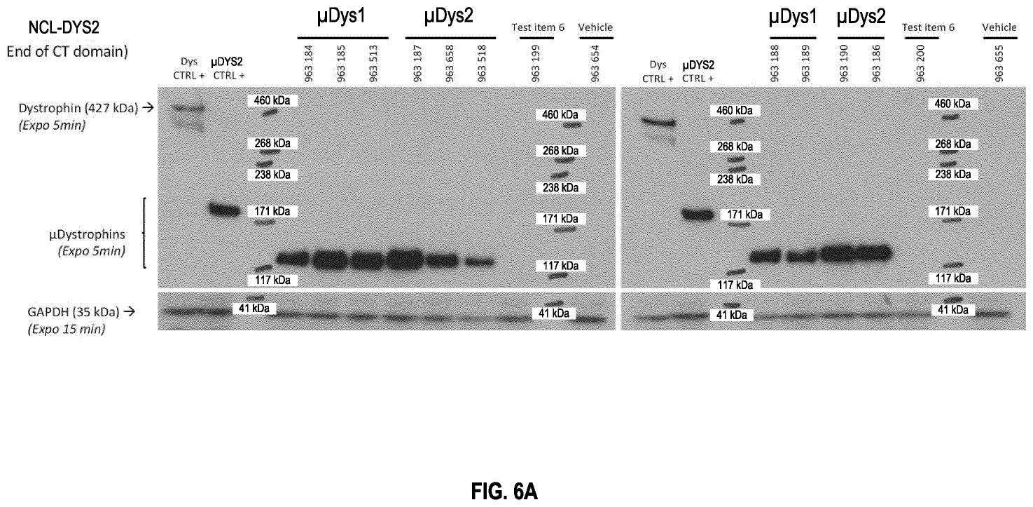

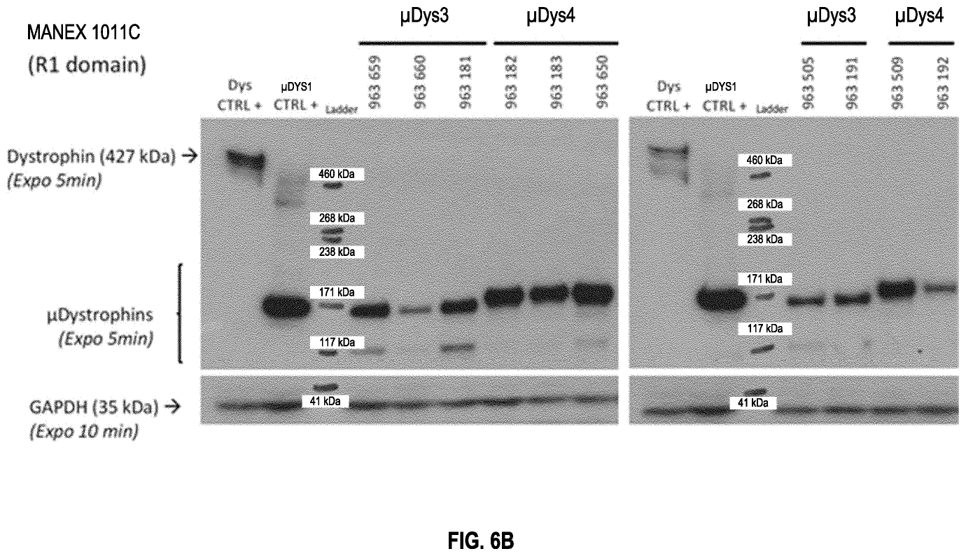

Microdystrophin protein expression in the heart after four weeks was analyzed using Western Blot and immunofluorescence (IF) assays, as shown in FIGS. 6A and 6B (Western Blots). For the Western Blot assays, 50 μg of total protein lysate (extraction with RIPA buffer) was loaded on a 3-8% tris-acetate gel (exposure time of 10 minutes).

### Example 6: Immunofluorescence

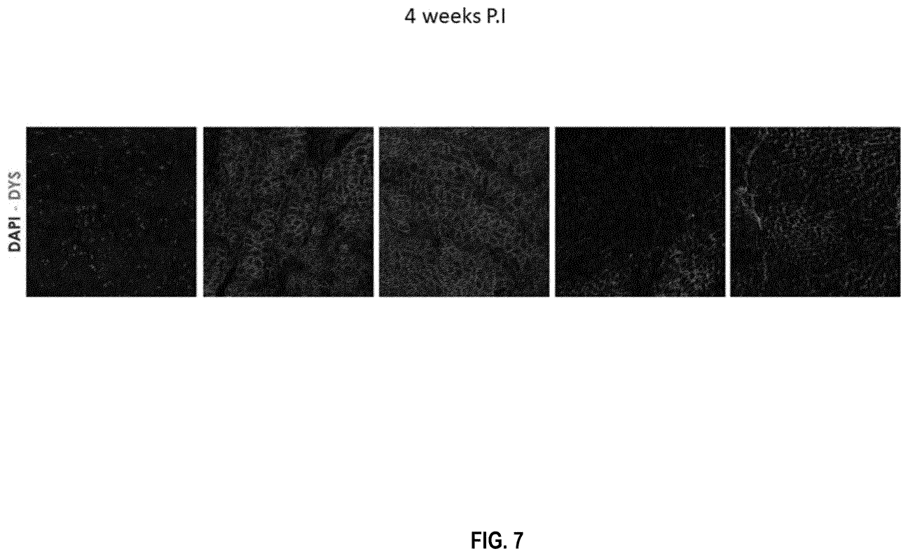

FIG. 7 is an immunofluorescene representation of microdystrophin expression in mdx^{4CV} mouse hearts.

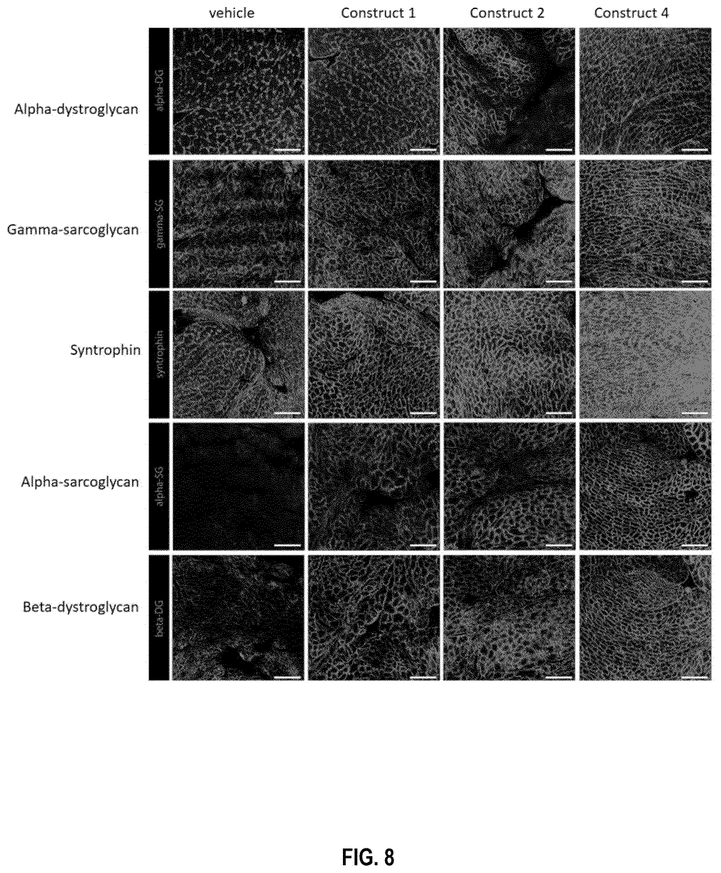

FIG. 8 depicts normal syntrophin expression in normal mouse heart, abnormal syntrophin expression in mdx^{4CV} mouse hearts, and restored syntrophin expression after gene therapy with microdystrophin Constructs 1, 2, and 4 in accordance with embodiments of the present disclosure. FIG. 8 further depicts restoration of the dystrophin-dystroglycan complex with increased expression of α-dystroglycan, β-dystroglycan, α-sarcoglycan and γ-sarcoglycan after gene therapy with microdystrophin Constructs 1, 2, and 4.

### Example 7: Gene Therapy Efficacy Study in DMD^{mdx} Neonatal Rats

A gene therapy efficacy study was performed on 80 Sprague Dawley male rats including rats with the DMD^{mdx} mutated dystrophin gene and the wild-type dystrophin gene. Table 2 shows the experimental parameters for this study. Rats were injected at 3-7 days old with vectors encoding microdystrophin constructs (AAV9-TNNT2-μDys2 or AAV9-TNNT2-μDys4), an empty vector, or the vehicle. The number of animals in each group was 8. At 3 and 6 months post-injection, cardiac function, biodistribution, RNA expression, protein expression and localization, and immunohistochemistry were assessed.

TABLE 2
Experimental Parameters for Study in DMD^{mdx} Neonatal Rates

| Group | Genotype | Test Item | Dose | Follow-Up |
|---|---|---|---|---|
| 1A | DMD^{mdx} | μDys2 | 2 × 10¹⁴ vg/kg | 3 months |
| 1B | DMD^{mdx} | μDys2 | 2 × 10¹⁴ vg/kg | 6 months |
| 2A | DMD^{mdx} | μDys4 | 2 × 10¹⁴ vg/kg | 3 months |
| 2B | DMD^{mdx} | μDys4 | 2 × 10¹⁴ vg/kg | 6 months |
| 3A | DMD^{mdx} | Empty vector | 2 × 10¹⁴ vg/kg | 3 months |
| 3B | DMD^{mdx} | Empty vector | 2 × 10¹⁴ vg/kg | 6 months |
| 4A | DMD^{mdx} | Vehicle | — | 3 months |
| 4B | DMD^{mdx} | Vehicle | — | 6 months |
| 5A | Wild-type | Vehicle | — | 3 months |
| 5B | Wild-type | Vehicle | — | 6 months |

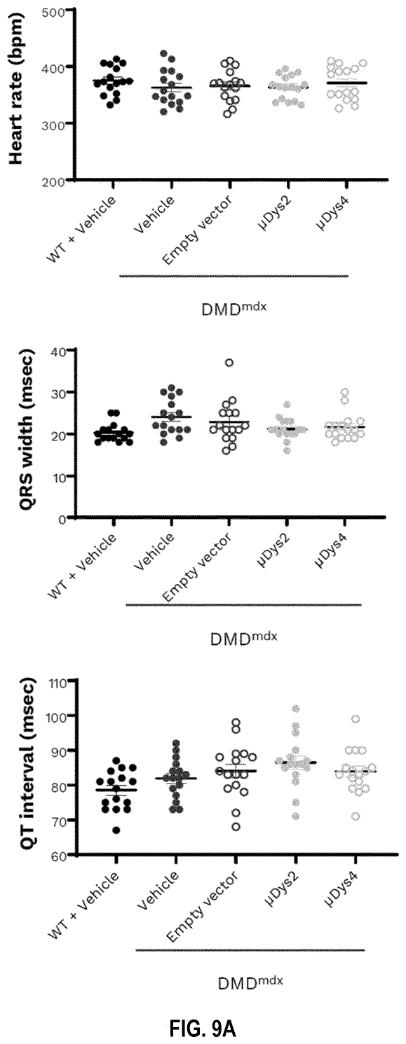

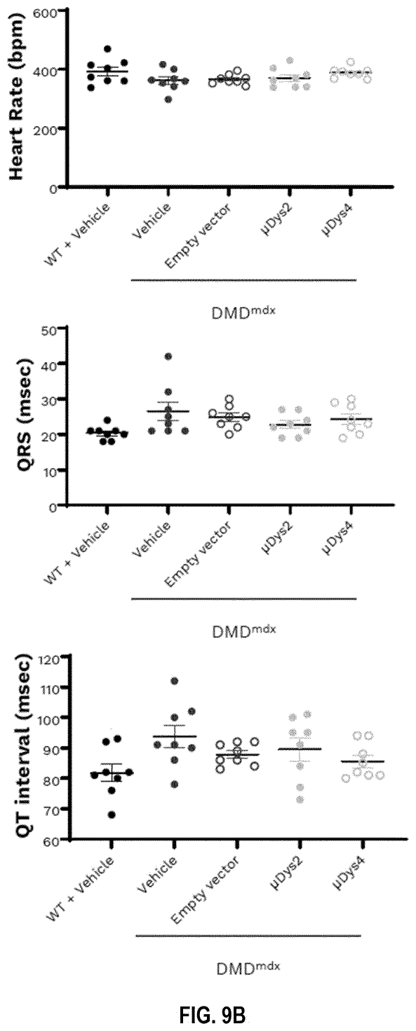

FIGS. 9A and 9B show plots of heart rate, QRS width, and QT interval derived from electrocardiograph measurements at three months post-injection (9A) and six months post-injection (9B), revealing a trend toward a therapeutic effect in reduced QRS and QT interval for the treated DIvD^{mdx} trats.

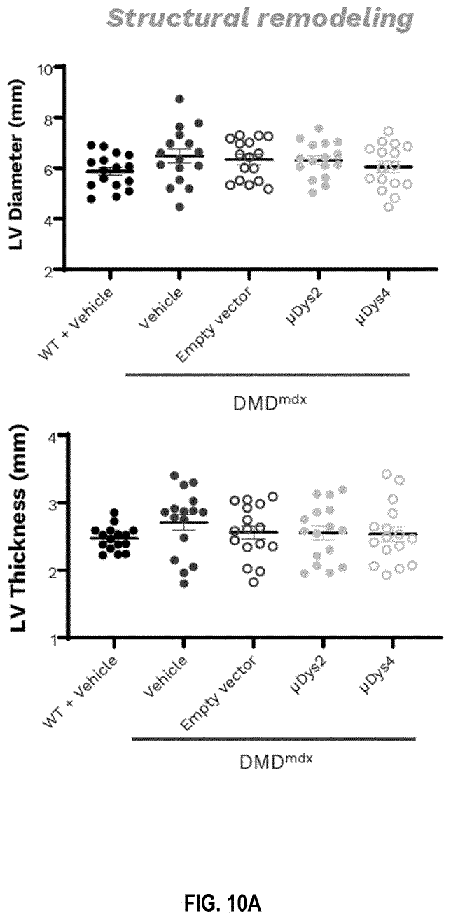

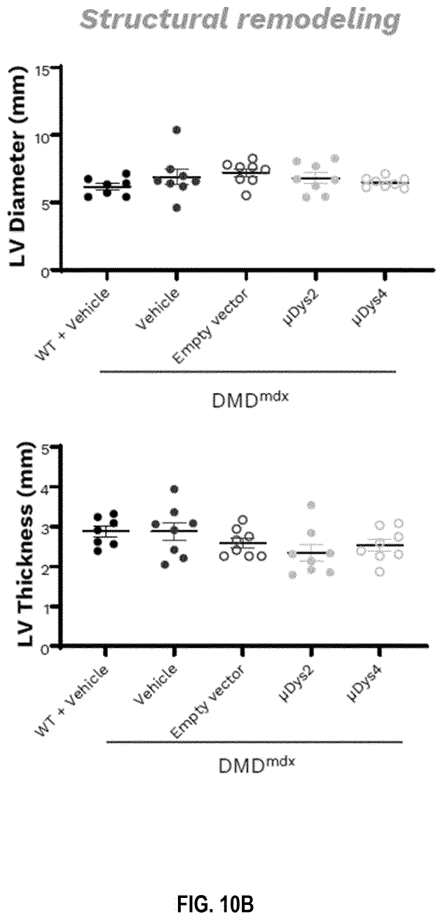

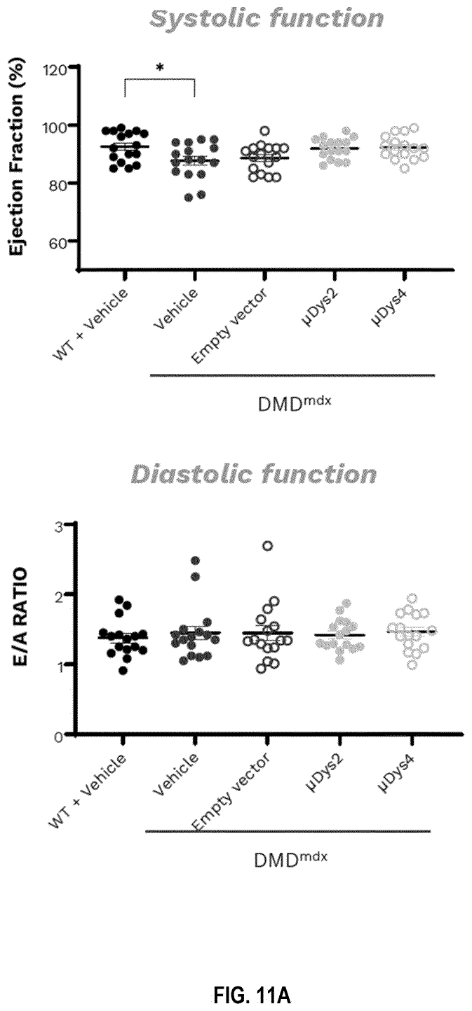

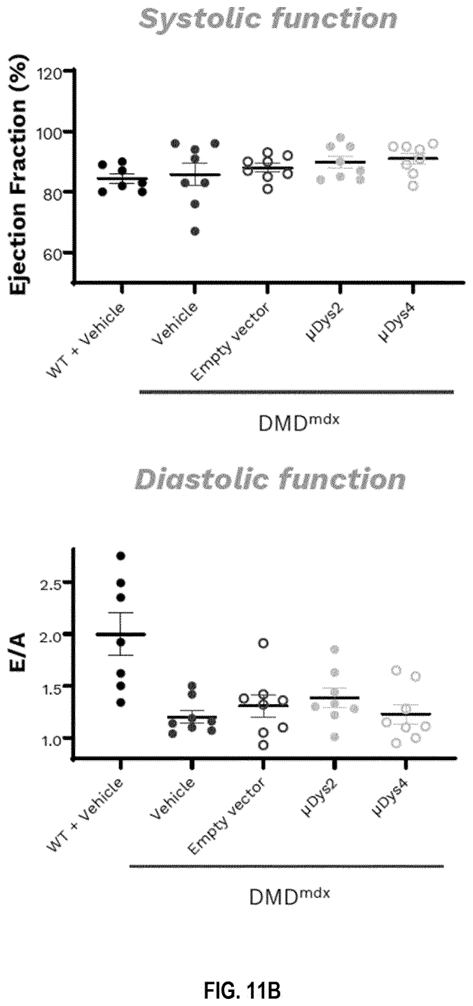

FIGS. 10A and 10B show structural remodeling of the left ventricle (LV), including plots of LV diameter and LV thickness, at three months post-injection (10A) and six months post-injection (10B). FIGS. 11A and 11B show plots of systolic function (ejection fraction) and diastolic function (E/A ratio) at three months post-injection (11A) and six months post-injection (11B). The data reveals that the microdystrophin-treated DMD^{mdx} rats tend to be more similar to wild-type rats, with ejection fraction being significantly altered at three months of age.

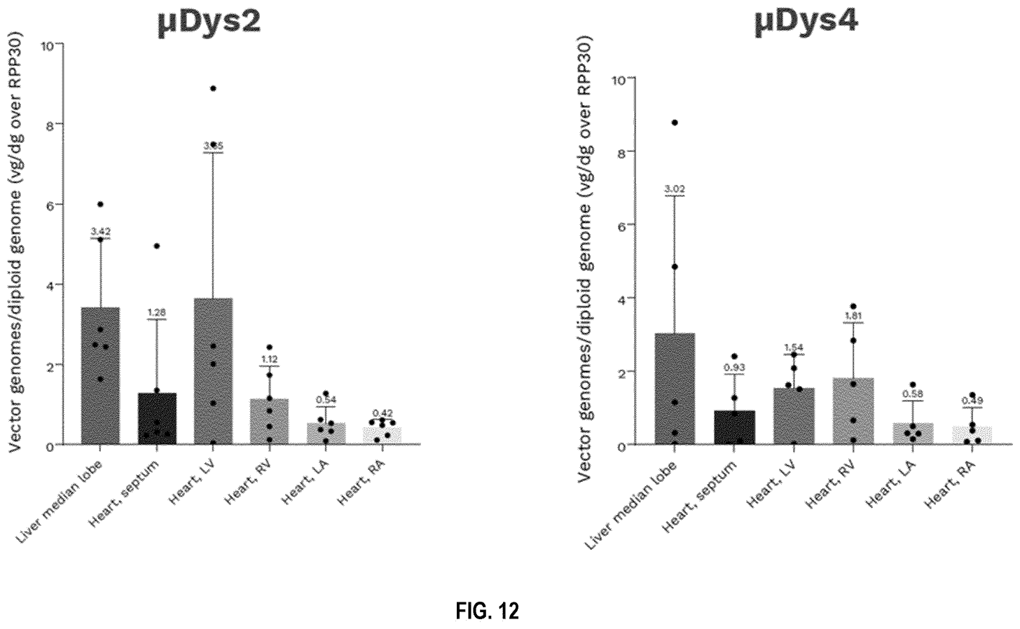

FIG. 12 shows plots of biodistribution (vg/dg) as measured in different areas of the heart and liver for both μDys2 and μDys4 vectors at three months post-injection, showing that both vectors lead to greater localization and transduction in the ventricles than in the atria. The measured vg/dl in the liver was likely a result of the delivery method (intraperitoneal injection).

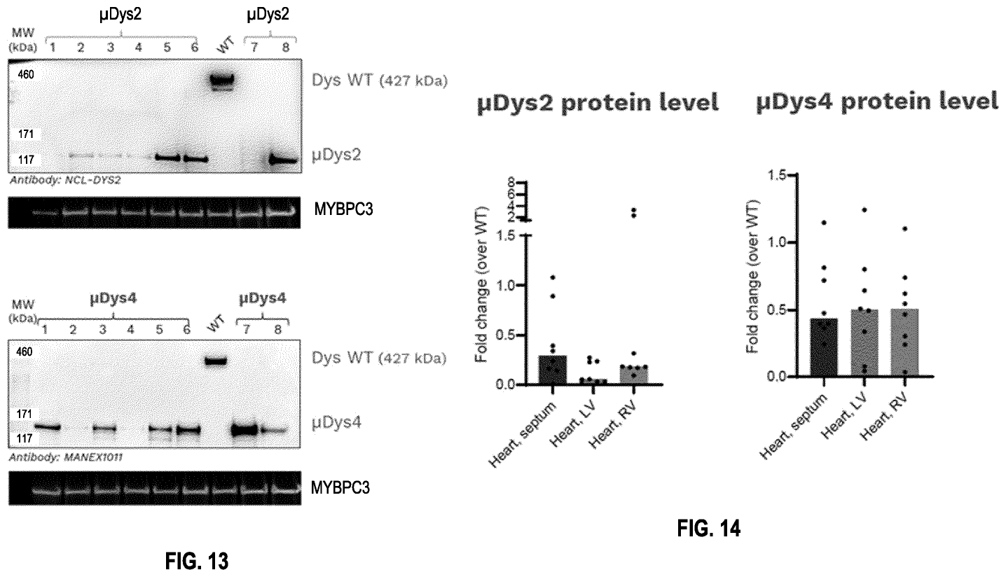

FIG. 13 shows western blot images assessing microdystrophin expression in the tissue of the left ventricles at three months post-injection. FIG. 14 shows expression levels of microdystrophin in the septum end ventricles of the heart, revealing homogeneous expression of μDys4 in the ventricles. Efficient transduction of the heart was observed for both vectors. Similar transgene expression in the ventricles and atria was observed, but more transcription of μDys4 was observed in the septum.

### Example 8: AAV9-microdystrophin Transduction in Engineered Heart Tissue

Two of the constructs (Constructs 2 and 4 corresponding to μDys2 and μDys4, respectively) were evaluated in engineered heart tissue (EHT). The EHT was cast with hiPSC-CM (cell link cpHet7% cTnT-positive cardiomyocytes), and transduced with the AAV9 constructs. Relaxation kinetics were measured in spontaneously contracting EHTs cultured in complete medium. In addition, 1- and 2-weeks after transduction, several relaxation parameters were evaluated under electrical stimulation at 1 Hz. Spontaneous beating frequency was analysed over time in complete and in serum-free DMEM media, at 2 weeks after transduction.

Some EHT cardiomyocytes were harvested at 14 days after transduction for protein extraction and further analysis. Exogenous protein levels were measured by Western blot using antibody against microdystrophin. Cardiac TnT protein level was used as reference.

EHTs function was analyzed without electrical stimulation with the use of the EHT test system equipped with CTMV.10 Software that provides standardized and robust video-optical evaluation of contractile force in a 24-well format as described in Hansen et al. (Development of a Drug Screening Platform Based on Engineered Heart Tissue, Circulation Research, vol. 107, no. 1, 2010, pages 35-44). EHTs were then measured under spontaneous beating conditions in complete medium over 4 weeks of culture.

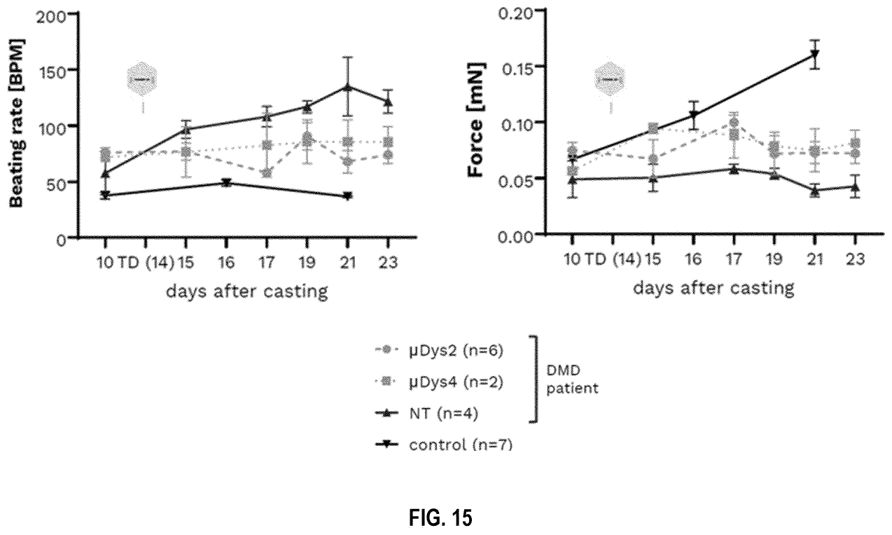

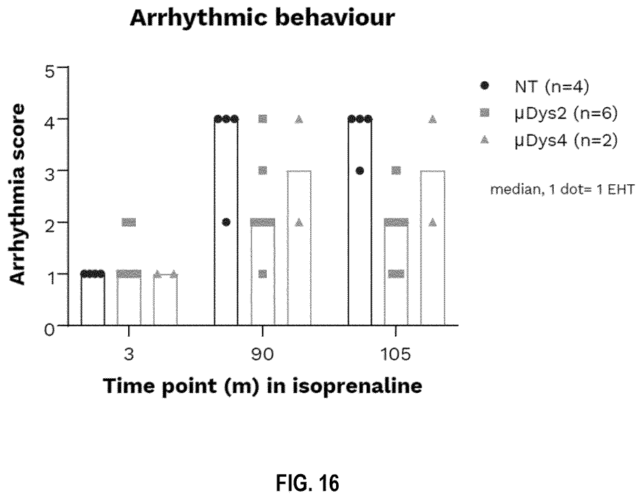

EHT measurement data were collected with CTMV.10 software and graphs were generated with the use of GraphPad Prism software. FIG. 15 shows resulting beating rate and force for treatment with μDys2 and μDys4, non-transduced (NT), and control samples (non-diseased isogenic controls), revealing that microdystrophins expression in transduced DMD EHTs stabilized the beating rate and improved force of contraction. FIG. 16 shows arrhythmia score, revealing that microdystrophin expression in transduced DMD EHTs can prevent arrhythmic events in response to andrenergic stimulation.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A gene therapy drug for treating or preventing cardiomyopathy in a human subject, the gene therapy drug comprising:
a vector comprising a polynucleotide coding sequence encoding for a microdystrophin protein, wherein the polynucleotide coding sequence encodes for a subset of repeat units R1-R24 of a dystrophin protein, and wherein the repeat units of the subset are selected from a group consisting of R1, R2, R24, portions thereof, and combinations thereof with the proviso that R1 and R24, or portions thereof, are present in the subset.

2. The gene therapy drug of claim 1, wherein the polynucleotide coding sequence encodes for only hinge 1, hinge 3, and hinge 4 of the hinge domains of the dystrophin protein.

3. The gene therapy drug of claim 1, wherein the polynucleotide coding sequence encodes for only hinge 1 and hinge 4 of the hinge domains of the dystrophin protein.

4. The gene therapy drug of claim 1, wherein the repeat units of the subset consist of R1 and R24, or portions thereof.

5. The gene therapy drug of claim 4, wherein the polynucleotide coding sequence encodes for the entire CT domain of the dystrophin protein.

6. The gene therapy drug of claim 4, wherein the polynucleotide coding sequence encodes for about 65% to about 85% of R1 and about 20% to about 40% of R24.

7. The gene therapy drug of claim 4, wherein the polynucleotide coding sequence encodes for about 65% to about 85% of R1 and about 50% to about 70% of R24 with the proviso that the polynucleotide sequence completely encodes exon 60 of the dystrophin gene.

8. The gene therapy drug of claim 4, wherein the polynucleotide coding sequence encodes for about 95% or greater of R1 and about 60% or less of R24 with the proviso that the polynucleotide sequence completely encodes exon 60 of the dystrophin gene.

9. The gene therapy drug of claim 1, wherein the polynucleotide sequence encodes for only exons 70-75, or portions thereof, of the cysteine-rich domain of the dystrophin protein.

10. The gene therapy drug of claim 9, wherein the polynucleotide sequence further encodes for at least a portion of repeat unit R2.

11. The gene therapy drug of claim 9, wherein the polynucleotide sequence encodes for only hinge 1, hinge 3, and hinge 4 of the hinge domains of the dystrophin protein, and wherein the repeat units of the subset consist of R1, R2, and R24, or portions thereof.

12. A gene therapy drug comprising a polynucleotide sequence comprising a microdystrophin-encoding sequence that is at least 90% identical to SEQ ID NO: 2, SEQ ID NO: 3, SEQ ID NO: 4, or SEQ ID NO: 5.

13. The gene therapy drug of claim 1, wherein the polynucleotide sequence further encodes a cardiac muscle-specific promoter, wherein the cardiac muscle-specific promoter is a TNNT2 promoter.

14. (canceled)

15. The gene therapy drug of claim 1, wherein the vector comprises a viral vector, and wherein the viral vector is an AAV vector.

16. (canceled)

17. The gene therapy drug of claim 15, wherein the AAV vector comprises AAV9.

18. The gene therapy drug of claim 1, wherein the product of expression of the polynucleotide coding sequence in the human subject is capable of restoring syntrophin binding and localization in dystrophin-deficient heart muscle.

19. A functional microdystrophin protein encoded by SEQ ID NO: 2 or a variant thereof having at least about 85% identity thereto, or having an amino acid sequence of SEQ ID NO: 25 or a variant thereof having at least about 85% identity thereto.

20. A functional microdystrophin protein encoded by SEQ ID NO: 3 or a variant thereof having at least about 85% identity thereto, or having an amino acid sequence of SEQ ID NO: 26 or a variant thereof having at least about 85% identity thereto.

21. A functional microdystrophin protein encoded by SEQ ID NO: 4 or a variant thereof having at least about 85% identity thereto, or having an amino acid sequence of SEQ ID NO: 27 or a variant thereof having at least about 85% identity thereto.

22. A functional microdystrophin protein encoded by SEQ ID NO: 5 or a variant thereof having at least about 85% identity thereto, or having an amino acid sequence of SEQ ID NO: 28 or a variant thereof having at least about 85% identity thereto.

23. A gene therapy drug to express the functional microdystrophin protein of claim 19 in cardiac tissue of a human subject.

24. A method of treating or preventing dystrophin-related cardiomyopathy in a human subject, the method comprising:
delivering a gene therapy drug of claim 1 to cardiac tissue of the human subject.

25. The method of claim 24, wherein the dystrophin-related cardiomyopathy is associated with Duchenne muscular dystrophy or Becker muscular dystrophy.
